(19) United States
(12) Patent Application Publication
(10) Pub. No.: US 2023/0277795 A1
(43) Pub. Date: Sep. 7, 2023

(54) Headgear Assembly for Securing a Fluid Tube

(71) Applicant: Masimo Corporation, Irvine, CA (US)

(72) Inventors: Jinwoo Kim, Los Angeles, CA (US); Chad A. DeJong, Los Angeles, CA (US); Mahdi Mohammadi, Mission Viejo, CA (US); Edmond Wing-Lun Yu, Chino Hills, CA (US); Jerrod Houston Tyler, Lake Forest, CA (US)

(21) Appl. No.: 18/177,418

(22) Filed: Mar. 2, 2023

Related U.S. Application Data
(60) Provisional application No. 63/324,278, filed on Mar. 28, 2022.
(60) Provisional application No. 63/316,211, filed on Mar. 3, 2022.

(51) Int. Cl. A61M 16/06

(52) U.S. Cl. CPC A61M 16/0683; A61M 16/0666

(57) ABSTRACT

Embodiments of the present disclosure provide a headgear assembly that includes a headband and at least one tube securement member coupled to the headband. The tube securement member includes channel having a first end and a second end. The channel is curved between the first and second ends and defines a receiving region configured to secure a portion of a tube connected to a nasal cannula. The channel faces away from skin of the subject when the headgear assembly is in use.

### RELATED APPLICATIONS

Any and all applications for which a priority claim is identified in the Application Data Sheet of the present application are hereby incorporated by reference under 37 CFR 1.57.

### FIELD

The present disclosure generally relates to headgear assemblies, and more particularly to headgear assemblies for securing a respiratory support device without using a subject's ears for support.

### BACKGROUND

Respiratory support devices are commonly used to deliver supplemental oxygen or increased airflow to subjects in need of respiratory assistance. A respiratory support device typically includes a lightweight fluid tube with two prongs arranged along a portion thereof that are inserted into the subject's nostrils. An end of the fluid tube is connected to an oxygen supply, such as a portable oxygen generator or a wall connection in a hospital. The respiratory support device is typically secured to the subject by looping portions of the fluid tube around the subject's ears.

### SUMMARY

Embodiments of the present disclosure provide a headgear assembly that offers a comfortable and effective way to secure a respiratory support device to a subject, without relying on the subject's ears for support. The headgear assembly includes a headband and tube securement members that are located at opposite ends of the headband. Each tube securement member includes a receiving region that securely retains the fluid tube. The configuration of the headgear assembly can promote proper positioning and alignment of the respiratory support device relative to the subject. By allowing for attachment of the respiratory support device to the subject without relying on the ears for support, the headgear assembly advantageously provides a more comfortable alternative to traditional around-the-ear configurations.

### BRIEF DESCRIPTION OF THE DRAWINGS

Throughout the drawings, reference numbers can be re-used to indicate correspondence between referenced elements. The drawings are provided to illustrate embodiments of the present disclosure and do not to limit the scope thereof.

FIG. 1 depicts an example respiratory support system in which a subject is shown wearing an example headgear assembly coupled to fluid tubes of the respiratory support system.

FIGS. 2A-2H depict first perspective, second perspective, front, back, first side, second side, top, and bottom views, respectively, of the headgear assembly of FIG. 1.

FIGS. 2I-2P depict first perspective, second perspective, front, back, first side, second side, top, and bottom views, respectively, in exploded format, of the headgear assembly of FIG. 1.

FIGS. 3A-3E illustrate top, back, front, side, and cross-sectional views, respectively, of the tube securement member of FIG. 2A.

FIG. 4 illustrates an example of the headgear assembly of FIG. 1 positioned on a subject.

FIGS. 5A-5C depict perspective, front, and side views, respectively, of the headgear assembly of FIG. 1 coupled to fluid tubes of an example respiratory support system and positioned on a subject.

FIG. 5D illustrates an example headgear assembly positioned on a subject.

FIGS. 6A-6C illustrate an example transitional sequence for attaching to a fluid tube to an example tube securement member.

FIGS. 7A-7H depict first perspective, second perspective, front, back, first side, second side, top, and bottom views, respectively, of an example headgear assembly.

FIGS. 8A-8E illustrate top, back, front, side, and cross-sectional views, respectively, of an example tube securement member of FIG. 7A.

FIG. 9 illustrates an example of the headgear assembly of FIG. 7A coupled to fluid tubes of an example respiratory support system and positioned on a subject.

FIGS. 10A-10H depict first perspective, second perspective, front, back, first side, second side, top, and bottom views, respectively, of an example headgear assembly.

FIGS. 11A-11D illustrate front, bottom, cross-sectional, and back perspective views, respectively, of the tube securement member of FIG. 10A.

FIGS. 12A and 12B illustrate example open and closed configurations, respectively, of the tube securement member of FIG. 10A.

FIG. 13 illustrates an example of the headgear assembly of FIG. 11A coupled to fluid tubes of an example respiratory support system and positioned on a subject.

### DETAILED DESCRIPTION

The present disclosure refers to a “respiratory support device,” which can encompass any device that delivers respiratory support and/or is used to monitor respiratory or other parameters. This includes, but is not limited to, nasal cannulas, oxygen masks, face tents, or other similar devices for delivering respiratory support via the nose or mouth, as well as capnographs or other monitoring devices for tracking respiratory or other parameters.

The present disclosure refers to a “respiratory support system” which can encompass any system that includes a respiratory support device. A respiratory support system can include one or more fluid tubes fluidically coupled to a respiratory support device. In addition or alternatively, a respiratory support system can include a fluid management apparatus that aids in the delivery of therapeutic fluids to and/or the collection of fluids from the subject.

Traditionally, nasal cannulas have been secured to a subject through a fluid tube looped around the ears, with the cannula nosepiece partially inserted into the nostrils, and the fluid tubes extending from the nasal cannula to the tops of and around the ears, and down together along the front of the chest to the oxygen supply in an “around-the-ear” configuration. While this design advantageously utilizes the subject's anatomical features to support and position the nasal cannula, the presence and weight of the fluid tubes around the ears can cause ear pain and discomfort. In addition, skin irritation is common on areas of the ears or face where tubing touches the skin.

To address these or other challenges, the present disclosure provides a headgear assembly that offers a more comfortable alternative to the traditional around-the-ear configuration for securing a respiratory support device to a subject. The headgear assembly includes a headband that facilitates attachment of the headgear assembly to the subject's head, without relying on the ears for support. The headband includes a pair of tube securement members positioned at opposite ends of the headband, adjacent to the subject's ears, that securely retain fluid tubes connected to the respiratory support device. Each tube securement member includes a receiving region that securely retains a respective portion of the fluid tube and introduces a kink-free bend into the fluid tube. The bend introduced by the tube securement members redirects the fluid tube towards the subject's neck or chest, such that the fluid tubes extend from the respiratory support device (e.g., nasal cannula) to the respective tube securement members, and then down together along the front of the chest. By redistributing the weight from the ears, as seen in traditional around-the-ear configurations, to the subject's head, the headgear assembly reduces or eliminates ear discomfort often associated with those designs. Additionally, when the headgear assembly is properly worn by the subject, the positioning of the fluid tube in the tube securement members relative to the subject's head facilitates proper alignment of the respiratory support device.

The headgear assembly can provide advantages over traditional respiratory support systems. For example, the headgear assembly adventurously promote kink-free tube securement by implemented a receiving region that avoids sharp bends in the fluid tube that can lead to kinking. In some cases, heated air or pressure passes through the tube, which can cause vibrations in the tube that might traditionally result in kinking of the tube, skin irritation, or discomfort for the subject. The headgear assembly advantageously reduces the likelihood of kinking, skin irritation, and discomfort for the subject. Furthermore, the tube securement member of the headgear assembly is designed to secure the nasal prongs in a way that is similar to a wrap-around ear design, but without using the ears as support, allowing for a secure and comfortable fit.

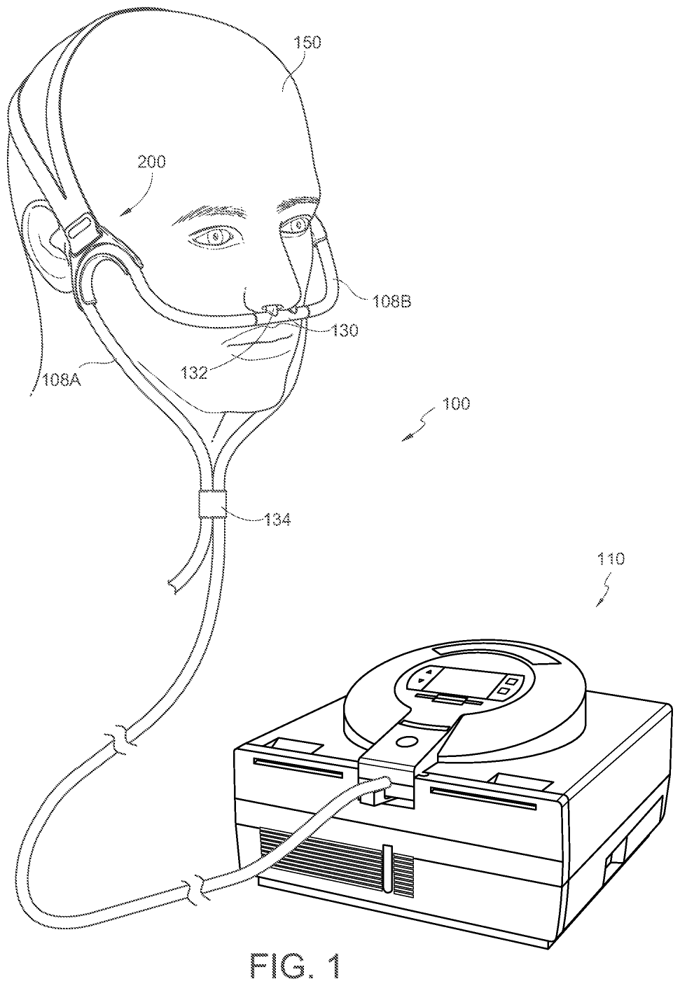

FIG. 1 depicts an example respiratory support system 100 in which a subject 150 is shown wearing an example headgear assembly 200 coupled to fluid tubes 108A, 108B of the respiratory support system 100. The respiratory support system 100 includes a fluid management apparatus 110, a respiratory support device 130, and fluid tubes 108A, 108B fludicially coupling the fluid management apparatus 110 and the respiratory support device 130. It will be appreciated that the respiratory support system 100 can include fewer, more, or different components, as desired. For example, as shown, the respiratory support system 100 can include a tube coupler 134 (for example, a slide bolo) for securing the fluid tube 108 under the chin of the subject 150. In some implementations, first ends of tubes 108A, 108B connect to ends of respiratory support device 130 (which can be a nasal cannula, for example), and second ends of tubes 108A, 108B connect to a single fluid tube that connects to fluid management apparatus 110, via a branched connector. Such a branched connector can have a first branch that connects to an end of tube 108A, a second branch that connects to an end of tube 108B, and a third branch that connects to a tube connect to and extending from fluid management apparatus 110. As another example, the respiratory support system 100 may include additional fluid tube, another fluid management apparatus, etc.

The fluid management apparatus 110 can facilitate the delivery of therapeutic fluids to the subject 150. For example, the fluid management apparatus 110 can deliver a variety of therapeutic fluids, such as oxygen or oxygen mixtures, to the subject as to provide ventilation and/or oxygenation. In some implementations, system 100 includes two fluid delivery tubes (such as tubes 108A, 108B) connected to a nasal cannula (for example, device 130) and joining one another at a three-way branched connector that connects to a third tube that connects to fluid management apparatus 110. In such implementations, therapeutic fluids can be delivered by the fluid management apparatus 110 through such third tube and through both of the tubes connected to the nasal cannula. In some implementations, the fluid management apparatus 110 includes a humidification source configured to provide humidity to accompany the therapeutic fluids. In some implementations, the fluid management apparatus 110 can facilitate the collection of fluids from the subject 150 for analysis or monitoring of the subject's exhaled and/or inhaled breathing gases, in addition to or as an alternative to providing therapeutic fluids to the subject 150. For instance, the fluid management apparatus 110 can deliver fluids through fluid tube 108B while concurrently collecting or receiving fluids through fluid tube 108A. In some implementations, system 100 includes a fluid delivery device (for example, apparatus 110) that provides therapeutic fluids (with or without humidification) via tube 108B and a first nasal prong 132 of cannula 130 and a gas sampling device that receives exhaled gases from tube 108A that in turn receives the exhaled gases from a second nasal prong 132 of cannula 130. In such implementations, the respiratory support system 100 may be utilized to receive exhaled gases from one nostril while delivering therapeutic gases to the other nostril. In some cases, the fluid management apparatus 110 can be implemented as a pumping air capnograph device. For example, the pumping air capnograph device can measure the amount of carbon dioxide (CO2) in a patient's exhaled breath by using a pump to draw in and expel air from the patient's airway and then analyzing the exhaled air for its CO2 content.

The respiratory support device 130 is depicted in this example as a nasal cannula, which can be utilized to deliver therapeutic fluids to and/or collect exhaled fluids from the wearer's nose. The nasal cannula includes one or more prongs 132 that are placed inside the wearer's nostrils and one or more tube connection portions that are coupled to the fluid tube 108. Although the design of the respiratory support device 130 may vary across embodiments, it may include any one or any combination of features of the cannula described in U.S. Pat. No. 10,441,196 (the “'196 patent”), issued Oct. 15, 2019, entitled “Nasal/Oral Cannula system and Manufacturing,” which is hereby incorporated by reference herein. In some implementations, the respiratory support device 130 may include an additional or alternate prong that is coupled to an oral breath collector and/or gas delivery system, such as the one described in the '196 patent that was previously incorporated by reference.

The headgear assembly 200 can securely attach to the head of the subject 150 and the fluid tube 108. When properly secured to both the subject's head and the fluid tube 108, the headgear assembly 200 can promote proper positioning of the respiratory support device 130, without relying on the subject's ears for support. For example, when the respiratory support device 130 is a nasal cannula, the headgear assembly 200 can secure the fluid tube and properly position prongs of the nasal cannula in the nostrils of the subject such that the nasal prongs are maintained in a substantially straight configuration (e.g., not “kinked”). Inhibiting “kinking” of nasal prongs within the subject's nostrils advantageously provides for uninterrupted flow in and/or out of the nostrils and minimizes or eliminates pressure fluctuations.

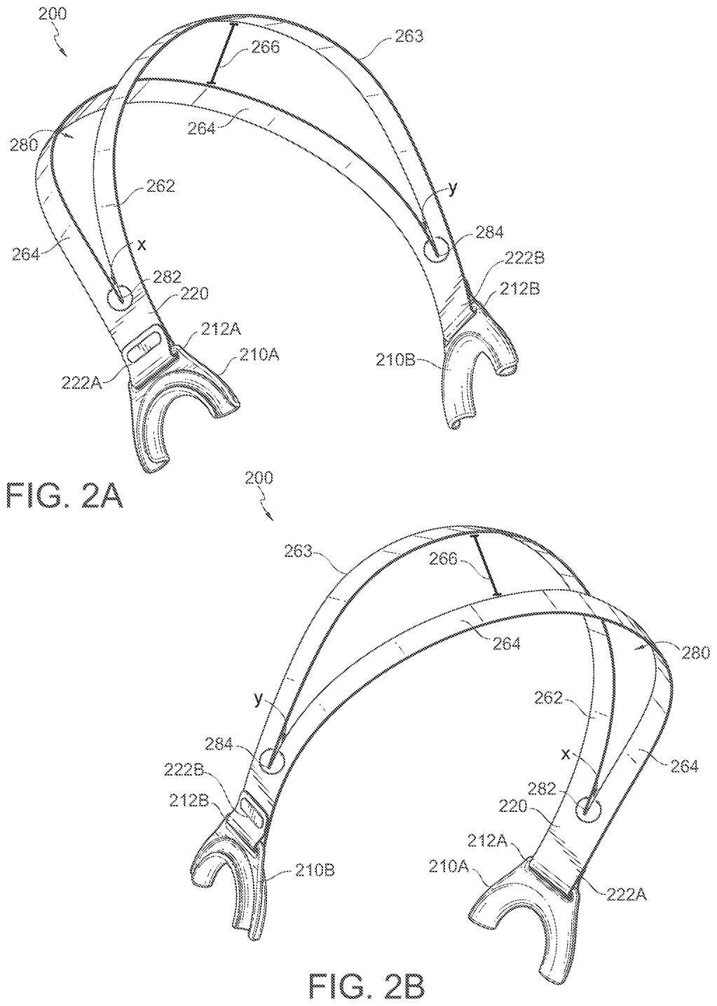

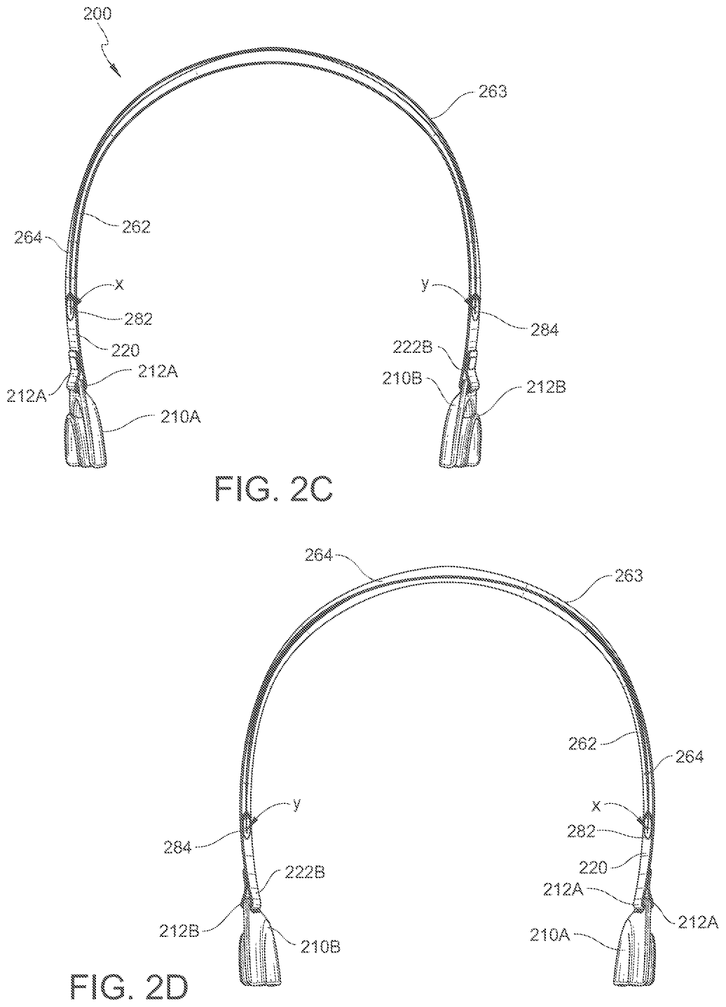

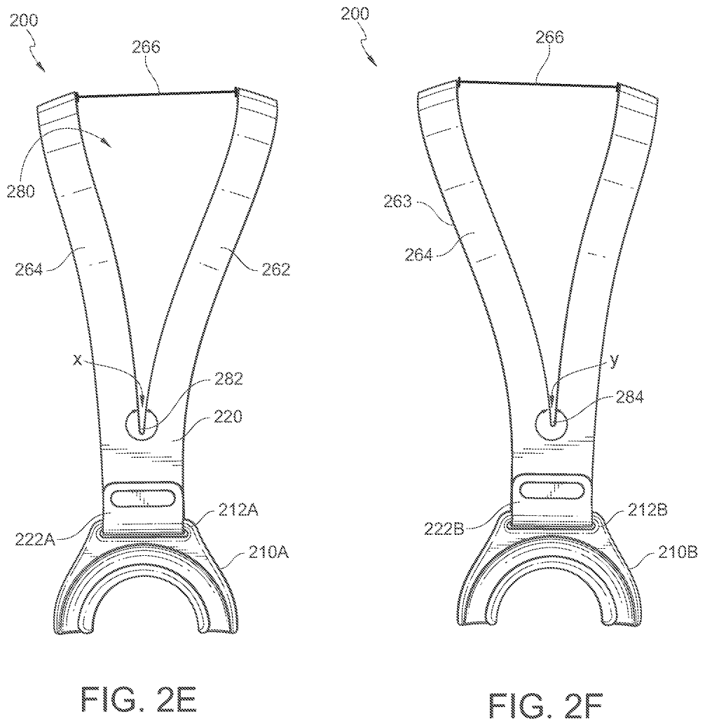

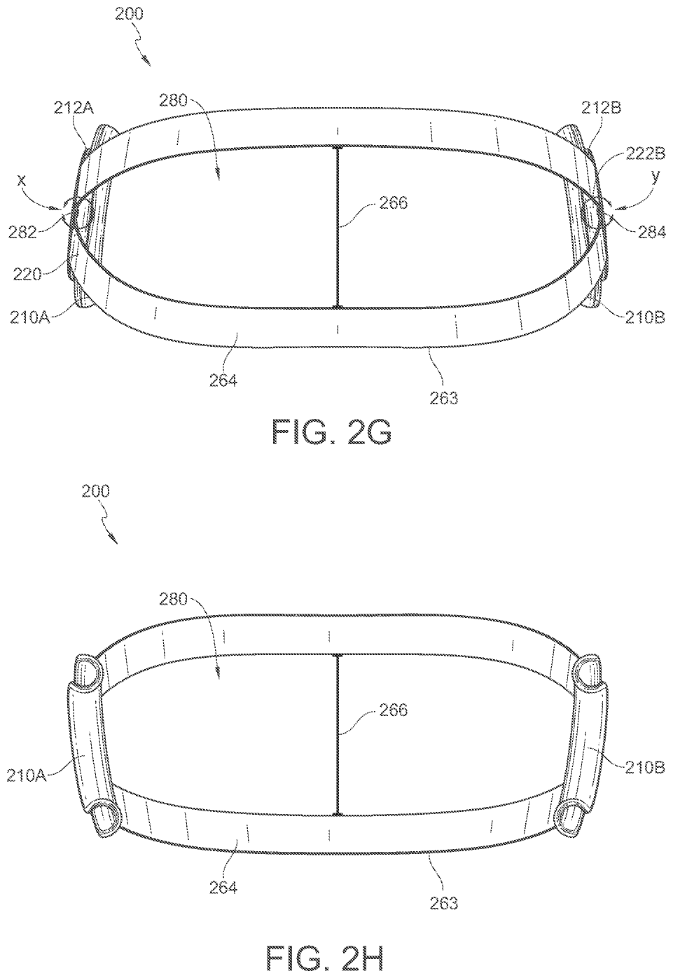

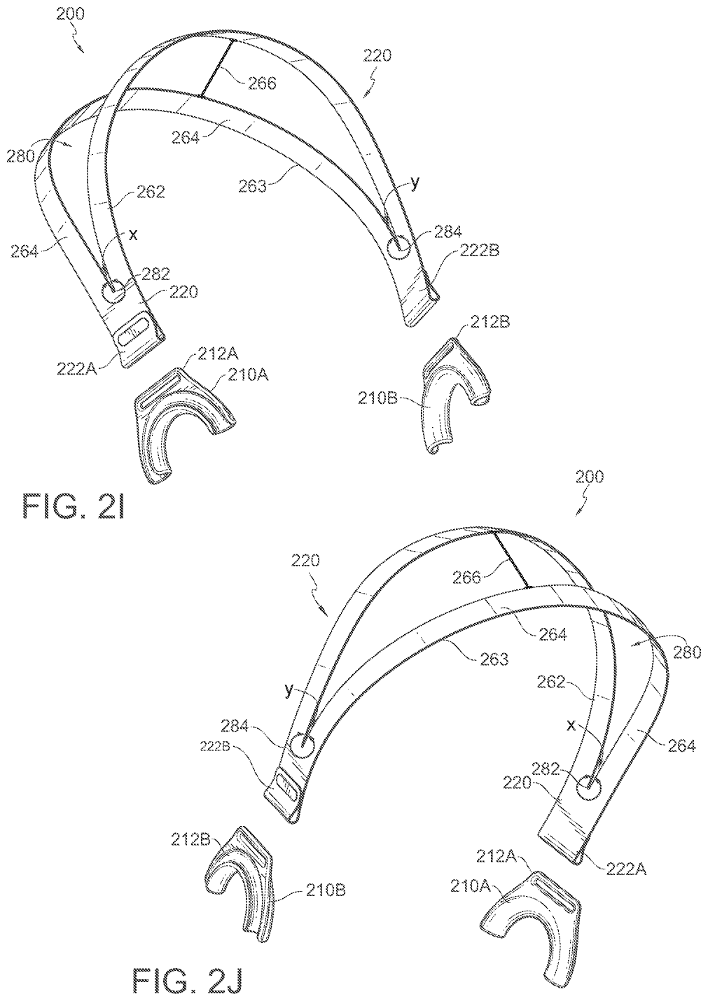

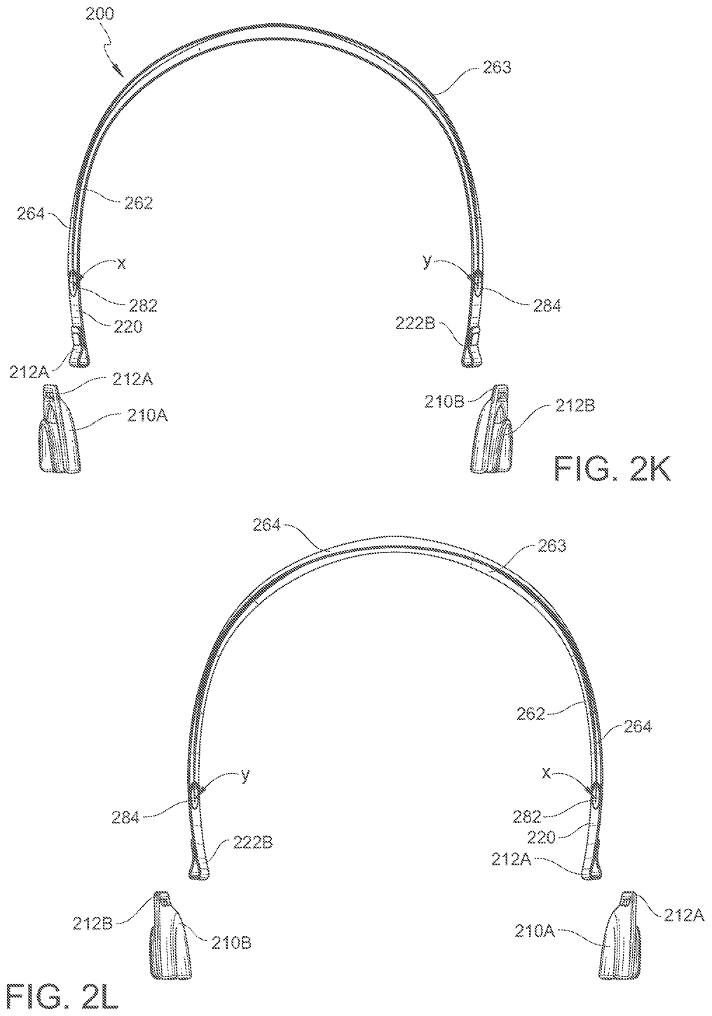

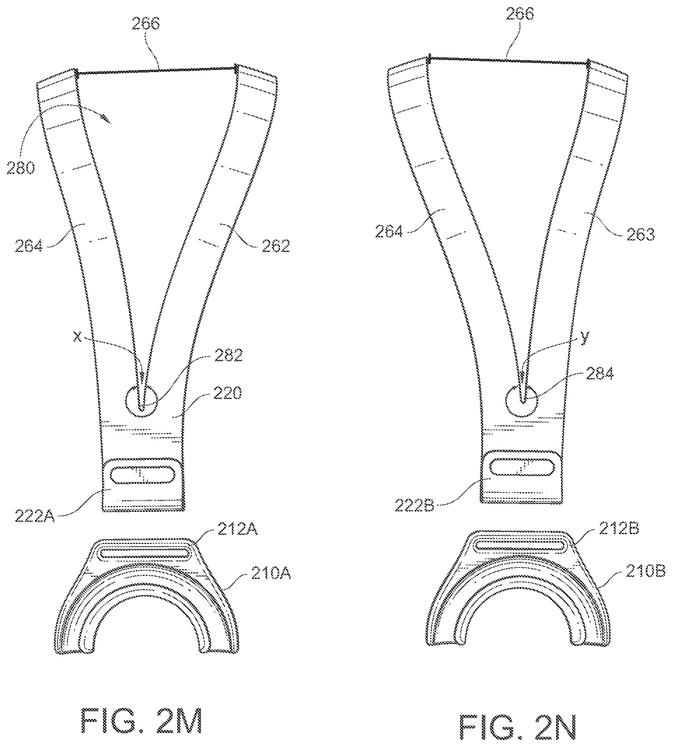

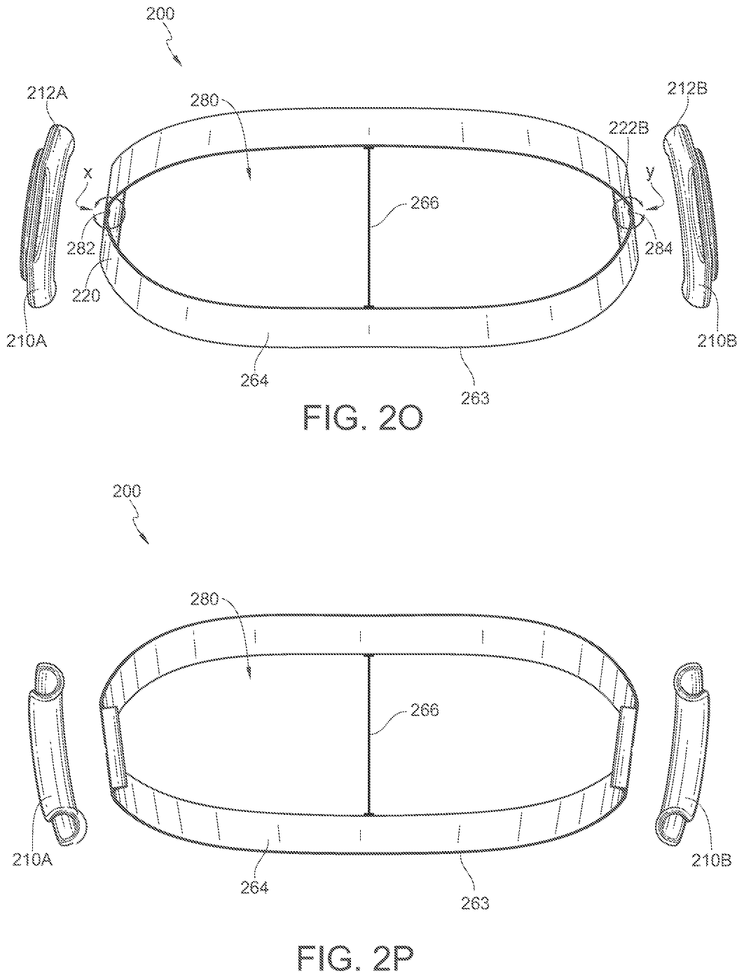

FIGS. 2A-2H depict first perspective, second perspective, front, back, first side, second side, top, and bottom views, respectively, of the headgear assembly 200 of FIG. 1. FIGS. 2I-2P depict first perspective, second perspective, front, back, first side, second side, top, and bottom views, respectively, in exploded format, of the headgear assembly 200 of FIG. 1.

The headgear assembly 200 includes a headband 220 and one or both of tube securement members 210A, 210B that are attached to either end of the headband 220. For ease of description, the tube securement members 210A, 210B may be referred to individually or collectively as the tube securement member(s) 210. However, the collective reference should not be interpreted as limiting their design or functionality. For example, in some cases, the tube securement members 210A, 210B may have different shapes, sizes, or features, or may be mirror images of each other. The headgear assembly 200 may include additional, different, or fewer components depending on the embodiment. For instance, a headgear assembly 200 may include a single tube securement member 210 and/or a headband 220.

The headband 220 separately engages with each of the plurality of tube securement members 210 to form the headgear assembly 200. The headband 220 can include a first engagement member 222A for coupling to an engagement member 212A of the first tube securement member 210A and can include a second engagement member 222B for coupling to an engagement member 212B of the second tube securement member 210B. For ease of description, the first and second engagement members 222A, 222B can be individually or collectively referred to as the engagement member 222, and the engagement members 212A, 212B can be individually or collectively referred to as the engagement member 212. In some cases, the engagement member 222 can be implemented as a strap and the engagement members 212 can be implemented as a slot. In some such cases, to join the tube securement member 210 to the headband 220, an end of the strap of the headband 220 can be inserted into the slot of the tube securement member 210, and the end of the strap can be folded back towards and secured to a portion of itself. In some cases, the strap may be adjustable so as to tighten or loosed the headband on the subject's head. In some implementations, a tab is utilized to secure the strap to itself. Such a tab can have a size and/or shape that is smaller than a size and/or shape of the strap (for example, a smaller width) and can secure (for example, removably secure) to an end of the strap and another portion of the strap.

In some implementations, the engagement members 212, 222 are associated with and/or represent connection points between tube securement members 210 and the headband 220. For example, any suitable engagement and/or locking features can be employed on the engagement members 212, 222. In some instances, the configuration of the engagement members 212, 222 is switched such that the engagement member 212 of the tube securement member 210 includes a strap and the engagement member 222 of the headband 220 includes a slot. As another example, the tube securement member(s) 210 and the headband 220 may be connected together via coinciding coupling features (e.g., a snap fit pairing, a strap and buckle, a living hinge and coinciding lip, an interference fit pairing, a hook and loop fastener, a protrusion and slot, a clip, a clamp, a clasp, a buckle, a button, a hook and protrusion, a screw and aperture, a wire, etc.). In some cases, more than one coupling feature can be used. Further still, in some cases, the headband 220 is connected to the tube securement member(s) 210 such that no coupling is required. For example, the headband 220 may be integrally molded with the tube securement member(s) 210.

The headband 220 can include one or more curved elongate bands to facilitate securement to the head of the subject. For example, the headband 220 includes a first elongate band 262 (sometimes referred to as a “front band”) and a second elongate band 264 (sometimes referred to as a “back band”). The front band 262 can include a contoured surface 263 (e.g., substantially matched to a shape of the head) extending from the first engagement member 222A to the second engagement member 222B. Similarly, the back band 264 can include a contoured surface extending from the first engagement member 222A to the second engagement member 222B. As shown, the front band 262 and the back band 264 are joined together at their ends and define a gap 280 therebetween that progressively increases in size towards the middle of the bands 262, 264 to a distance 266. The gap 280 between the bands 262, 264 can facilitate the securement of the headband 220 to the head of the subject. For example, the gap 280 can enable the front band 262 to be positioned on a top or top/front portion of the subject's head, and the enable the back band 264 to be positioned on a top or top/back portion of the subject's head.

The front band 262 and the back band 264 can form an angle, X, at a first juncture 282 and an angle, Y, at a second juncture 284. The angles, X and Y, may be the same or different and can vary across embodiments. In some cases, one or both of the angles, X, Y, are acute angles, such as less than about 45 degrees, less than about 30 degrees, less than about 15 degrees, etc. In some cases, one or both of the angles, X, Y, are right angles or obtuse angles. The size of the gap 280, length of the distance 266, and or size of the angles X, Y, can vary across embodiments. In some cases, the gap 280, distance 266, and/or angle X, Y, can be based on a subject's head size, and/or may be adjustable. Furthermore, in some cases, the gap 280 and/or distance 266 is a function of the angles X, Y. For example, as one or both of the angles, X, Y increase, so does the distance 266 and/or the gap 280. The headband 220 can be made any suitable material. For example, the headband 220 can be made of fabric and/or foam. In some cases, the headband 220 is made of a resilient, shape memory material. For example, the headband 220 can have the ability to return from a deformed state (temporary shape) to its original (permanent) shape when induced by an external stimulus, such as stretching associated with being worn on the subject's head. As another example, the headband 220 may be made of a rigid material, such as plastic, which is capable of providing a biasing force. For example, the headband 220 can include an open-loop headband having an arcuately contoured one-piece configuration terminating at a pair of legs for resiliently capturing therebetween the head of the wearer. In some cases, the headband 220 is made of at least one of a soft fabric material, an elastic material, or a non-elastic material. The headband 220 can be implemented differently across embodiments. For example, in some cases, the headband 220 is implemented as one or more of a single-strand headband, a multi-strand hairband, a scarf, a hat, a hair clip, glasses, or any suitable structure for supporting one or more of the tube securement members 210 on the subject's head. In some cases, the headband 220 is a unitary member. In some cases, the front band 262 and/or back band 264 may be separate pieces.

When the headgear assembly 200 is secured to the subject 150, the headband 220 includes a subject-facing surface (which may also be referred to as a “first surface” or an “inward-facing surface”) and a non-subject facing surface (which may also be referred to as a “second surface” or an “outward-facing surface”). The subject-facing surface can include one or more features to facilitate attachment and/or slip of the headgear assembly 200. For example, the subject-facing surface can include multiple grip teeth for reducing the likelihood of sliding. In some cases, the non-subject facing surface can be smoother than the subject-facing surface.

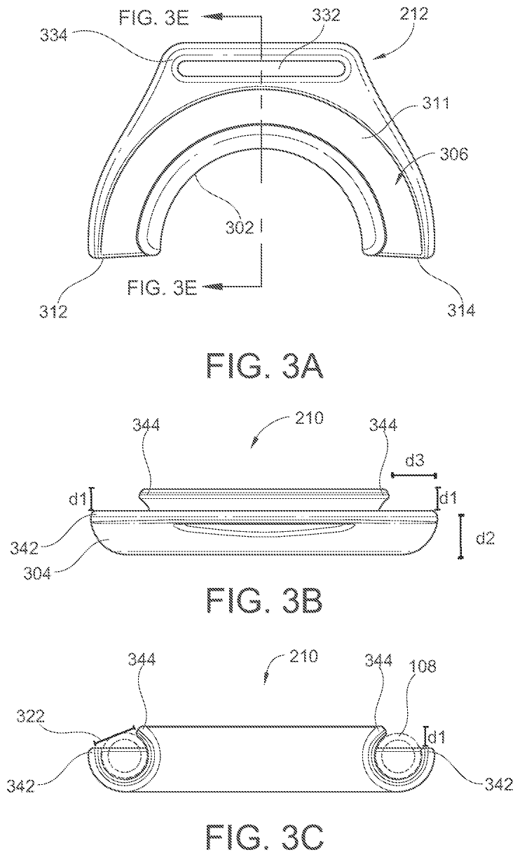

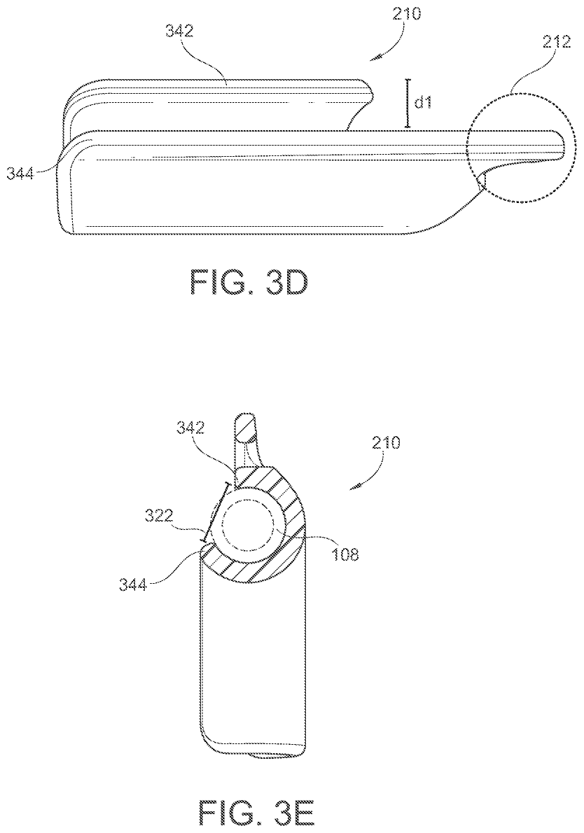

FIGS. 2I-2P illustrate various views of the headband 220 detached from tube securement members 210A, 210B. FIGS. 3A-3E illustrate top, back, front, side, and cross-sectional views, respectively, of the tube securement member 210 of FIG. 2A. As shown, the tube securement member 210 includes a wall 302 and an engagement member 212. The wall 302 has a curved shape and includes a first end 312, a second end 314, and an interior surface 311. The interior surface 311 of the wall 302 defines a channel defining receiving region 306 for removably receiving a portion of fluid tube 108. The wall 302 and/or receiving region 306 can be configured to secure a portion of fluid tube 108 such that, when the portion of the fluid tube 108 is secured within the receiving region 306, the portion of the fluid tube 108 has a curved configuration. The receiving region 306 can extend from the a first end 312 of the wall 302 to the second end 314 of the wall 302.

The receiving region 306 can be a channel, for example, extending continuously between such first and second ends 312, 314. For example, the channel can be curved between the first and second ends 312, 314 and defines the receiving region 306. In some cases, the channel faces away from skin of the subject when the headgear assembly is in use. In some cases, the channel faces towards skin of the subject when the headgear assembly is in use. The first and second ends 312, 314 can be referred to as ends of the receiving region 306, ends of the channel, or ends of the wall 302. The wall 302 can extend circumferentially around the receiving region 306. For example, the wall 302 can enclose a majority of a cross-sectional perimeter of the receiving region 306. In some cases, the wall 302 encloses less than about 50%, more than about 50%, 60%, 70%, 80, or 90%, between about 55% to about 85%, between about 65% to about 75%, or between about 60% to about 85% of a cross-sectional perimeter of the receiving region 306.

The wall 302 can include a first edge 342 and a second edge 344 that each extend along a length of the wall 302. The second edge 344 can be inset from (which also may be referred to as “inwardly spaced from”) the first edge 342 by a distance, d3. Accordingly, the first edge 342 may be referred to as an “outer edge” of wall 302 and the second edge 344 may be referred to as an “inner edge” of wall 302. In some cases, the first edge 342 defines all or a portion of an exterior perimeter of the curved elongate wall 302, and/or the second edge 344 defines all or a portion of an interior perimeter of the curved elongate wall 302. As shown, when laying on a subject-facing side of the tube securement member 210, the second edge 344 can have a first height, d2, and can be higher than the first edge 342, such as by a gap, d1. The gap, d1, and distance, d2, and height, d3, can vary across embodiments. For example, d1, d2, or d3 may range from a millimeter (mm) to a few millimeters, or from a few millimeters to a centimeter, to up to a few centimeters. As another example, the size of d1, d2, or d3 may be relative to the height of the tube securement member 210. For example, d1, d2, or d3 may be about ½, about ⅓, about ¼, about ⅕, about ⅛, etc. the size of the height of the tube securement member 210. Alternatively, in some cases, the second edge 344 and the first edge 342 are the same or a substantially similar height. Furthermore, in some implementations, the first edge 342 is higher than second edge 344.

In some cases, the first edge 342 is situated along, or lies along, a first plane, while the second edge 342 is situated along, or lies along, a second plane. In some cases, the first plane is spaced apart from the second plane, such that the distance between the two planes defines the magnitude of gap, d1. In some cases, the first plane and the second plane are parallel to each other or slightly off-parallel.

The wall 302 defines an opening 322 extending continuously along a length of the receiving region 306. The opening 322 is defined by a gap between first and second edges 342, 344 of the wall 302. The first and second edges 342, 344 can be spaced apart by a predetermined distance that is less than a diameter of the receiving region 306. The opening 322 allows a portion of fluid tube 108 to be inserted into and/or removed from receiving region 306. The opening 322 can extend along all or a portion of a length of the receiving region 306. The tube securement members 210 can be secured to the subject's head (for example, via the headband 220 such that the opening 322 (and portion of the tube secured therewithin) faces toward or away from the subject's skin).

As shown, the receiving region 306 be a generally curved or bent region so as to introduce a kink-free bend into the fluid tube 108 when the fluid tube 108 is secured within the receiving region 306. For purposes of this disclosure, the expression kink-free bend is broadly interpreted to include any bend in the fluid tube 108 that does not completely restrict the flow of fluid through the fluid tube 108. In some cases, a kink-free bend is a bend that does not substantially impede the flow of fluid through the fluid tube 108. In some cases, the curve or bend of the receiving region 306 is a gradual curve, such as a round curve. In some cases, the curve or bend of the receiving region 306 is a tight curve or bend. In some cases, the receiving region 306 bends or curves the fluid tube 108 so as to redirect the fluid tube 108 toward the ground, chest, neck, etc.

In this example, the receiving region 306 (and thus a portion of the wall 302) is generally cylindrical and/or is sized and/or shaped to accommodate a generally cylindrical shaped tube. For purposes of this disclosure, the expression “generally cylindrical” is intended to indicate that the exterior structure of the receiving region 306 has the geometrical shape of a cylinder; however, it also encompasses the case in which the entire receiving region 306, or a portion thereof, is curved or bent along its length. The receiving region 306 may additionally or alternatively include other geometric shapes, for example a geometric figure whose two end faces (e.g., the first end 312 and the second end 314) are similar and whose side is curved or bent along its length. In some such cases, the end faces can form the shape a circle, an oval, a half or partial circle or oval, a triangle, a rectangle, a square, a polygon, or the like.

Although the opening 322 is illustrated as outwardly facing, it will be understood that the location of the opening 322 may vary across embodiments. For example, in some cases, the opening 322 is inward facing (e.g., facing towards the subject) such that the fluid tube 108 can be inserted through a bottom portion of the tube securement member 210. For purposes of this example, the bottom portion of the tube securement member 210 can refer to the skin-contact portion of tube securement member 210 when secured to the subject 150. As another example, the opening 322 may extend along an outer edge or an inner edge of the tube securement member 210. As another example, the opening 322 may be partially outwardly facing and partially downward facing. For example, the opening 322 may follow a helical pattern so as to introduce a spiral structure to the tube securement member 210.

In some implementations, the tube securement member 210 can include one or more disconnection-resisting features or structures configured to resist disconnection between the fluid tube 108 and the tube securement member 210. The disconnection resisting features can be configured to initially require a higher level of force to initiate disconnection then, once that force is overcome, lower force to allow disconnection of the fluid tube 108 from the tube securement member 210 without additional frictional resistance. The disconnection resisting features can similarly initially require a higher level of force to initiate connection of the fluid tube 108 with the tube securement member 210. As an example, the disconnection-resisting features can include at least a portion of the opening 322 being sized smaller than the diameter of the fluid tube 108. In this way, to connect the fluid tube 108 to (or disconnect it from) the tube securement member 210, sufficient force must be applied to expand the opening 322 to allow the fluid tube 108 to pass. As such, the tube securement member 210 can resist undesired disconnections of the fluid tube 108 from the receiving region 306. Furthermore, in some cases, the first and second edges 342, 344 of the wall 302 each include a lip that extends toward the interior surface 311 of the wall 302. The lips can be disconnection-resisting features which apply a compressive force to the portion of the fluid tube to aid retainment of the portion of the fluid tube in the receiving region 306.

As another example, in some embodiments, the receiving region 306 can be sized and/or shaped to fit the fluid tube 108. Example sizes for fluid tubes range from 14 to 22 gauge or from 2 to 8 mm in external diameter. In some cases, the receiving region 306 is similarly sized to the exterior diameter of the fluid tube 108 such that the fluid tube 108 fits securely (e.g., snuggly) into receiving region 306. In some cases, the secure fit of the fluid tube 108 into receiving region 306 can prevent or limit lateral or rotational movement of the fluid tube 108 within the receiving region 306. In some cases, the tube securement member 210 (e.g., the wall 302) is configured to removably secure a portion of the fluid tube therein by frictional engagement between the interior surface 311 of the wall and an outer surface of the portion of the fluid tube.

The tube securement member 210 can be made any suitable material. In some cases, the tube securement member 210 is made of a resilient, shape memory material. For example, the tube securement member 210 can have the ability to return from a deformed state (temporary shape) to its original (permanent) shape when induced by an external stimulus, such as stretching associated with inserting or removing the fluid tube into or from the receiving region 306. In some cases, the tube securement member 210 is made of a rigid material, such as plastic.

The tube securement member 210 can include an engagement member 212 for engaging with the headband 220. The engagement member 212 can be integrally formed with the wall 302 or may be a separately attached piece. For example, the engagement member 212 may extend outwardly from a generally central portion of the wall 302. In addition or alternatively, the engagement member 212 may extend from another portion of the wall 302, such as the first end 312 or the second end 314. In this example, the engagement member 212 is implemented as a protrusion 334 and slot 332.

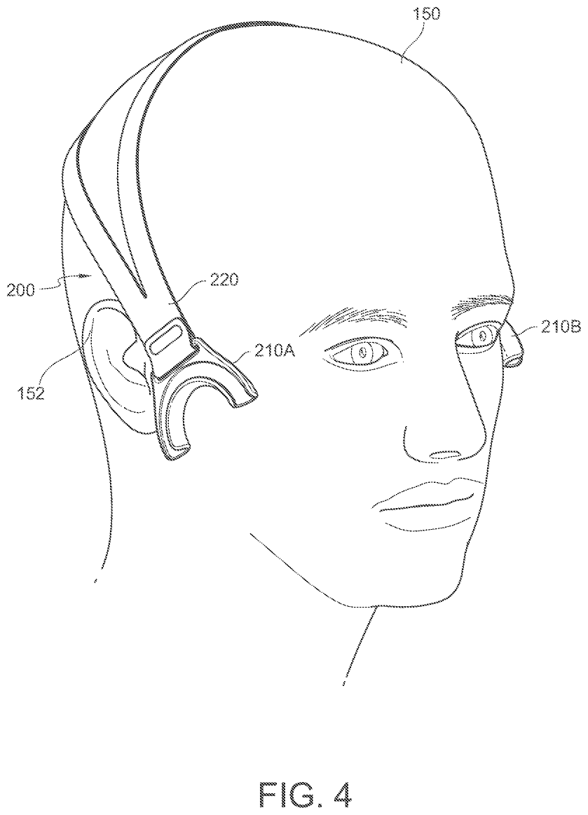

FIG. 4 illustrates an example of the headgear assembly 200 of FIG. 1 positioned on a subject 150. As shown, the headgear assembly 200 can be positioned such that the tube securement members 210 are adjacent to, but not supported by, the ears 152 of the subject 150. For example, when positioned on the subject 150, the headband 220 can reside on an upper and/or upper rear of the subject's head and the tube securement member 210 can reside in a location that is adjacent to the ears. Although the illustrated example shows the tube securement member 210 in front of the ear, in some cases, the tube securement member 210 can be location behind, above, or below the ear. The tube securement members 210 can cooperate to support the fluid tubing during use. For example, the tube securement members 210 can cooperate to properly position prongs of a nasal cannula in nostrils of the subject 150.

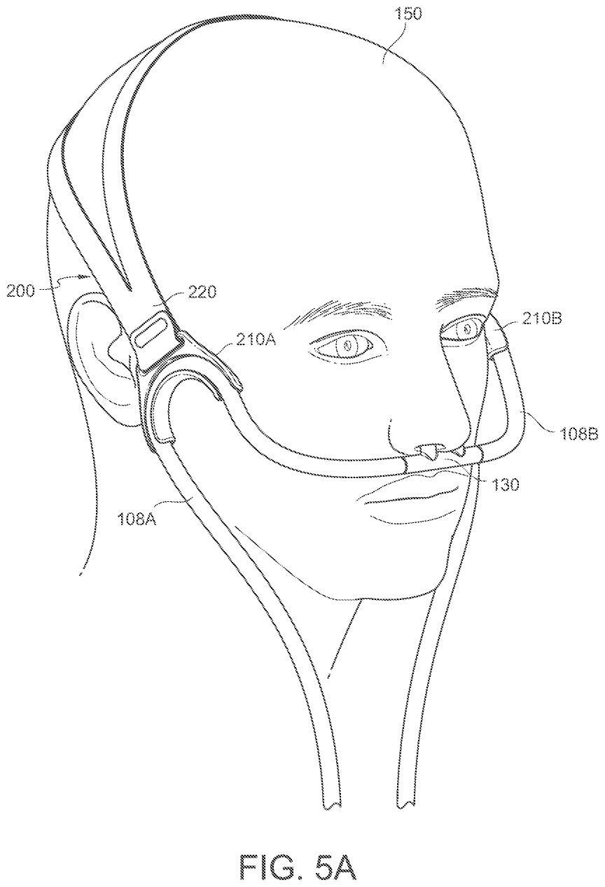

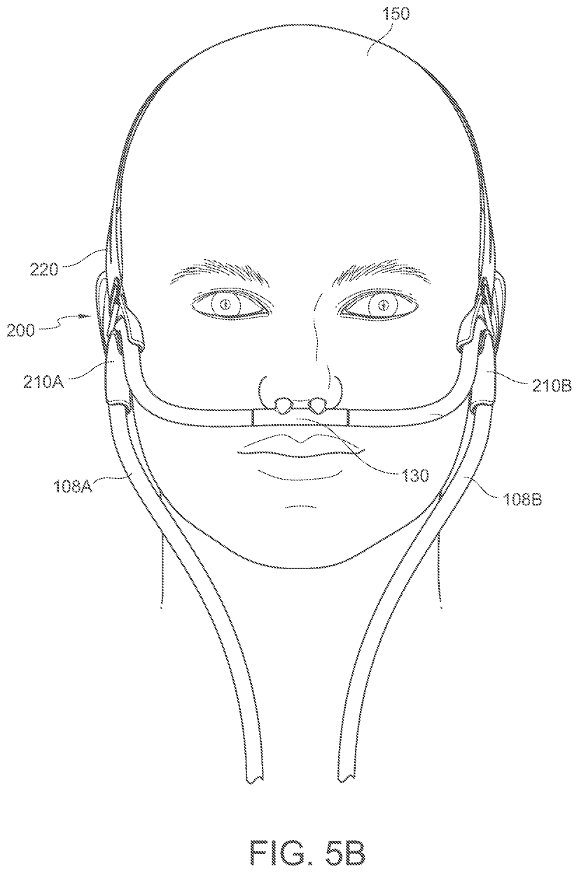

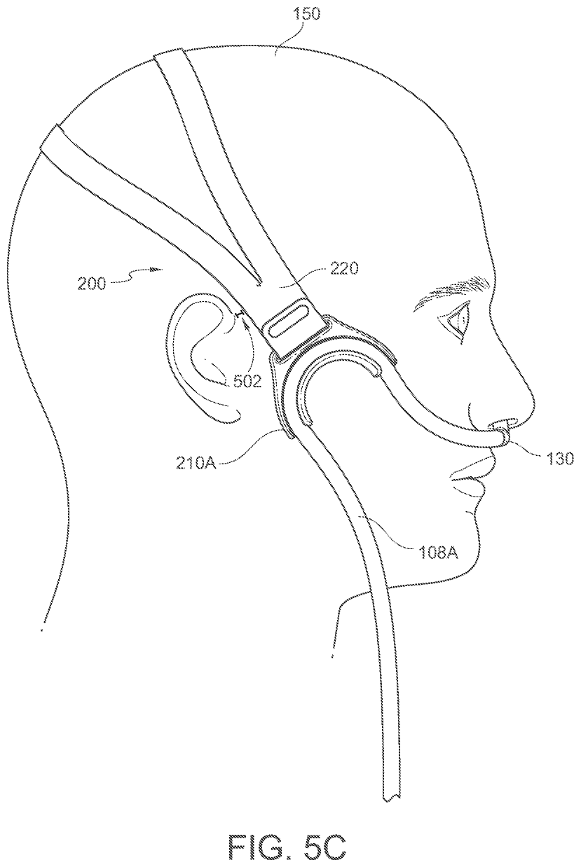

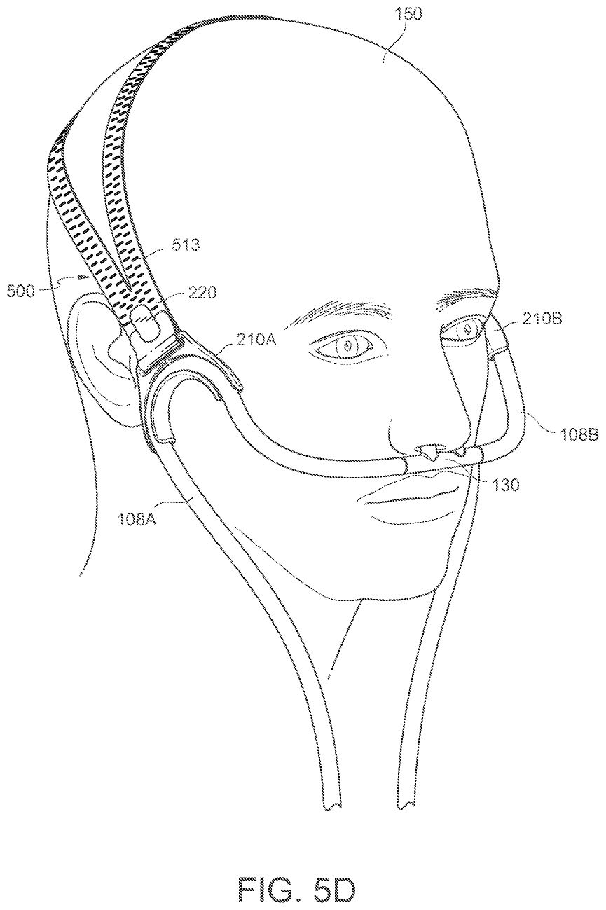

FIGS. 5A-5C depict perspective, front, and side views, respectively, of the headgear assembly of FIG. 1 coupled to fluid tubes 108A, 108B of an example respiratory support system and positioned on a subject 150. As shown, when the headgear assembly 200 and respiratory support device 130 are attached to the subject, the fluid tubes 108 extend from the respiratory support device 130 to the respective tube securement members 210. The tube securement members 210 curve or bend the fluid tubes 108 down together along the front of the chest of the subject 150.

In some cases, the headgear assembly 200 does not contact any portion of the ear when worn by the subject 150. For example, as shown in FIG. 5C, when positioned properly on the subject 150, there may be a gap 502 between ear and the headgear assembly 200. The size of the gap 502 can vary across embodiment and may depend on the size and/or shape of the subject's head. In some cases, the size of the gap 502 falls within the range of a few millimeters to a few centimeters or more. By separating the headgear assembly 200 from the ear, the headgear assembly 200 advantageously provides a more comfortable system than conventional around-the-ear designs. In some implementations, the headgear assembly 200 is configured to secure to a subject's head and secure one or more portions of fluid tubes 108A, 108B (which can be connected to nasal cannula with one or more nasal prongs) such that no portion of the headgear assembly 200 contacts the subject's ears. For example, in some implementations, the headgear assembly 200 is configured to secure to a subject's head and secure one or more portions of fluid tubes 108A, 108B (which can be connected to nasal cannula with one or more nasal prongs) such that no portion of the headband 220 and/or no portion of the tube securement member(s) 210A, 210B (either or both of which can be included in assembly 200) contact the subject's ears. With reference to at least FIG. 5A, in some implementations, tube securement member(s) 210A, 210B are sized and/or shaped to secure and/or operably position portions of tubes 108A, 108B in a manner that is similar to a manner in which such tubes 108A, 108B would be secured and/or operably positioned if tubes 108A, 108B were wrapped around the subject's ears. For example, in some implementations (such as that illustrated in FIG. 5A), tube securement member(s) 210A, 210B are configured to secure portions of tubes 108A, 108B in a curved arrangement that is similar to a curved arrangement that such portions of tubes 108A, 108B would have if wrapped around the subject's ears. In some implementations, the headgear assembly 200 is configured to secure to a subject's head and secure one or more portions of fluid tubes 108A, 108B (which can be connected to nasal cannula with one or more nasal prongs) such that each tube securement member 210A, 210B is positioned: in front of the subject's ear; between the subject's ear and the subject's eye; and/or adjacent the subject's cheek (see FIGS. 5A 5C). In some implementations, the headgear assembly 200 is configured to secure to a subject's head and secure one or more portions of fluid tubes 108A, 108B (which can be connected to nasal cannula with one or more nasal prongs) such that tubes 108A, 108B do not contact skin of the subject's face (for example, cheek).

FIG. 5D illustrates an example headgear assembly 550 positioned on a subject. The headgear assembly 500 includes a headband 520 and a plurality of tube securement members 510 coupled to either end of the headband 520. The headgear assembly 500, tube securement members 510, and/or headband 520 can be embodiments of the headgear assembly 200, tube securement members 210, and/or headband 220 of FIG. 1. As compared to the headgear assembly 200 of FIG. 1, the headgear assembly 500 includes a different implementation of the headband 520. Here, the headband 520 of the headgear assembly 500 includes grip members 513 patterned along an exterior surface of the headband 520. The grip members 513 can additionally or alternatively be positioned along an interior surface of the headband 520. These grip members 513 can provide a more secure fit and prevent the headgear assembly 500 from slipping during use. Additionally, the headband 520 can include an adjustable strap 515 that allows for a customizable fit. For example, the strap 515 may be velcro-ed or reattachable by other means, allowing the user to detach an edge 517 of the strap 515, pull it tighter or loosen it as needed, and reattach the strap 515. This adjustability ensures that the headgear assembly 500 can fit a wide range of head sizes comfortably and securely, making it accessible for individuals with varying needs.

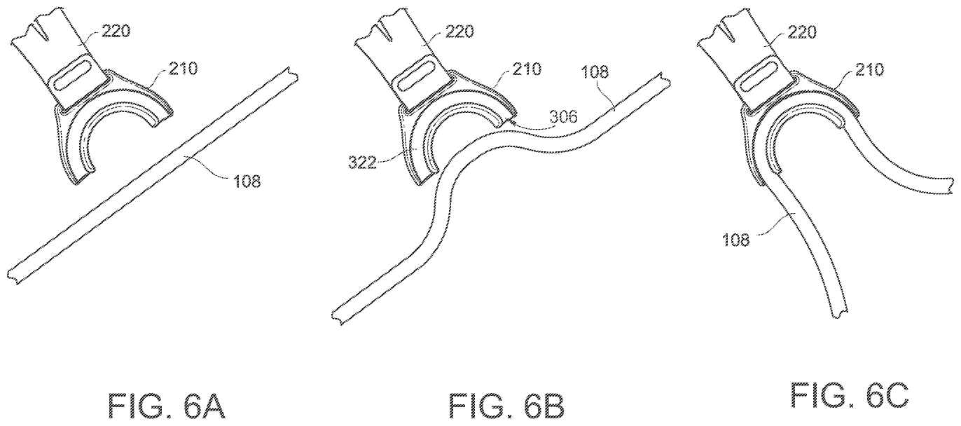

FIGS. 6A-6C illustrate an example transitional sequence for attaching a fluid tube 108 to the headgear assembly 200 of FIG. 1. In FIG. 6A, the headgear assembly 200 and the fluid tube 108 are provided to the subject. The subject can choose to wear the headgear assembly 200 prior to attaching to the fluid tube 108 or may connect the fluid tube to the headgear assembly 200 prior to putting it on. In FIG. 6B, a middle portion of the fluid tube 108 is bent so as to approximately match the shape of the receiving region 306. In this example, the receiving region 306 is generally C-shaped. In FIG. 6C, the middle portion of the fluid tube 108 is inserted into the receiving region 306 through the outward facing opening 322. In particular, the middle portion of the fluid tube 108 is generally aligned with the receiving region 306, and a force is applied along the length of the middle portion of the fluid tube 108 in the direction of the receiving region 306. As described herein, as part of a disconnection resisting feature, the width of the opening 322 may be smaller than or equal to the diameter of the fluid tube. In this way, the headgear assembly 200 advantageously resists disconnection of the fluid tube 108 from the receiving region 306.

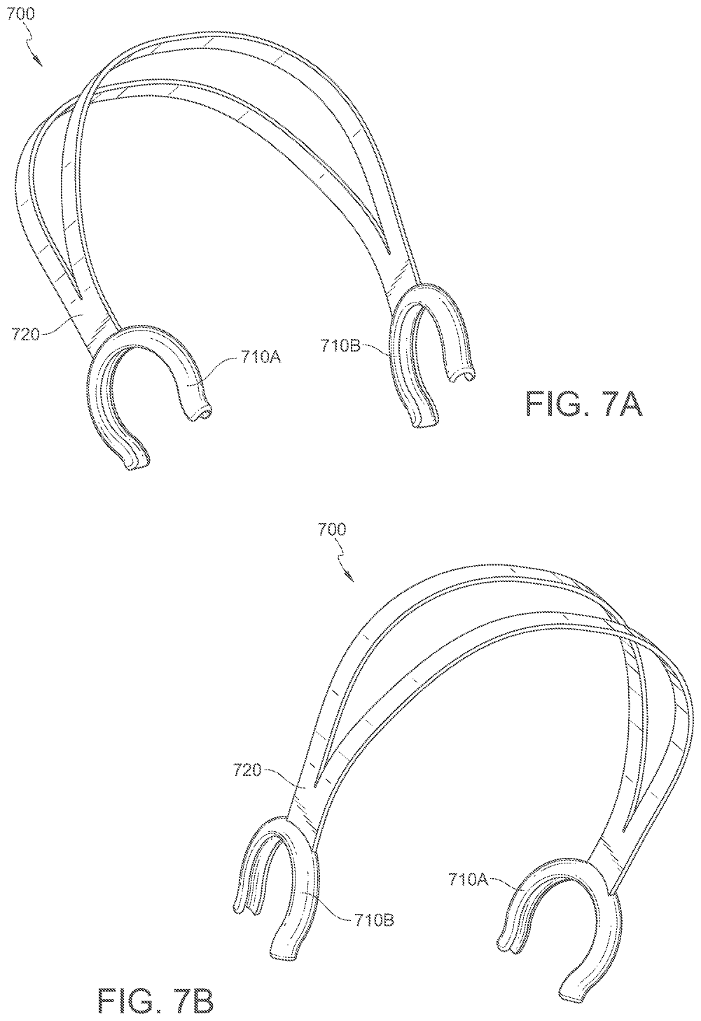

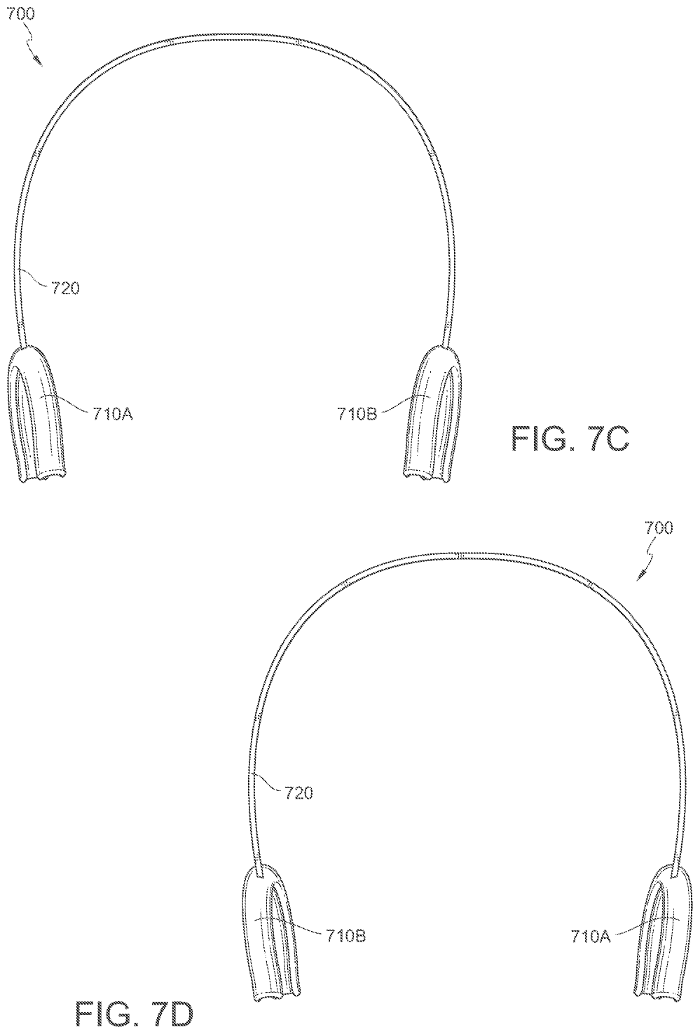

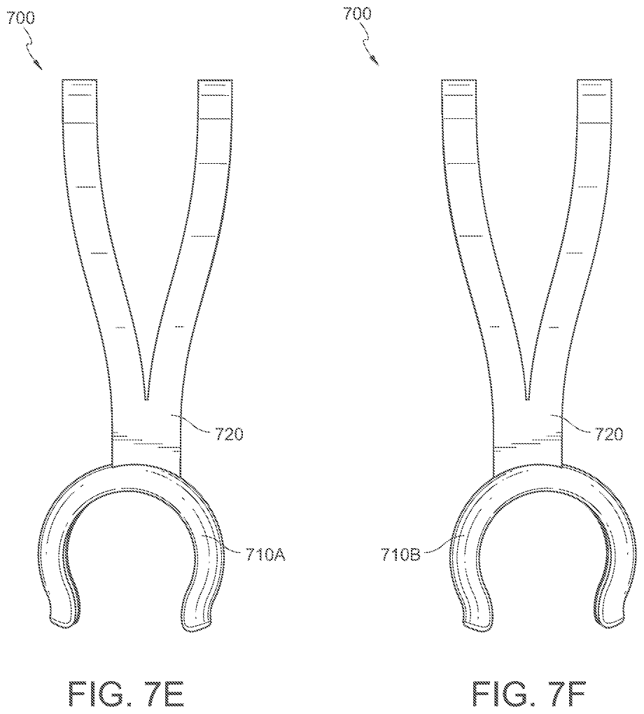

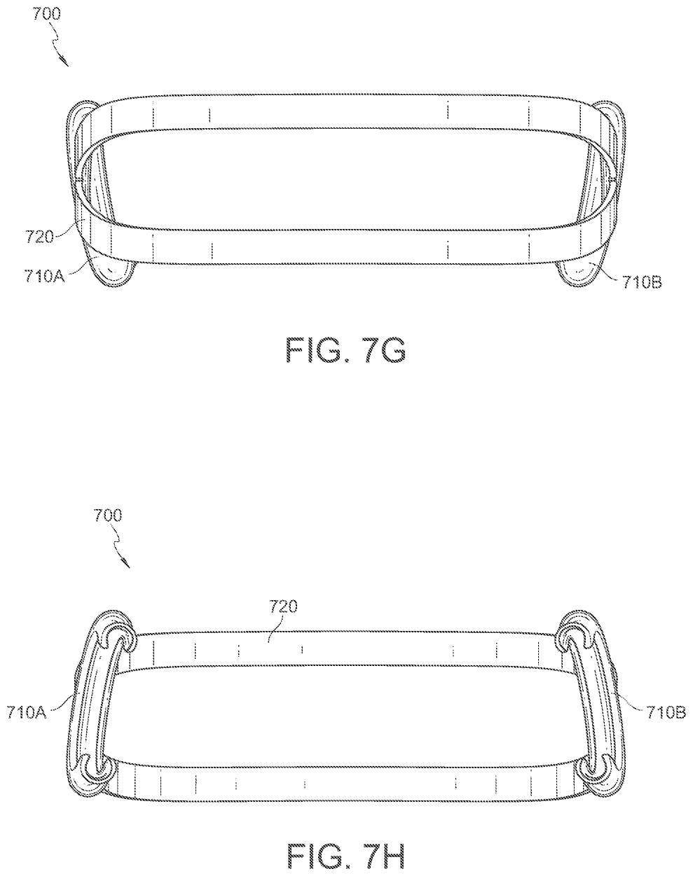

FIGS. 7A-7H depict first perspective, second perspective, front, back, first side, second side, top, and bottom views, respectively, of an example headgear assembly. The headgear assembly 700 includes a headband 720 and a plurality of tube securement members 710A, 710B coupled to either end of the headband 720. For ease of description, the plurality of tube securement members 710A, 710B are individually or collectively referred to as the tube securement member 710, the tube securement member(s) 710, or the tube securement members 710. The headgear assembly 700, tube securement members 710, and/or headband 720 can be embodiments of the headgear assembly 200, tube securement members 210, and/or headband 220 of FIG. 1.

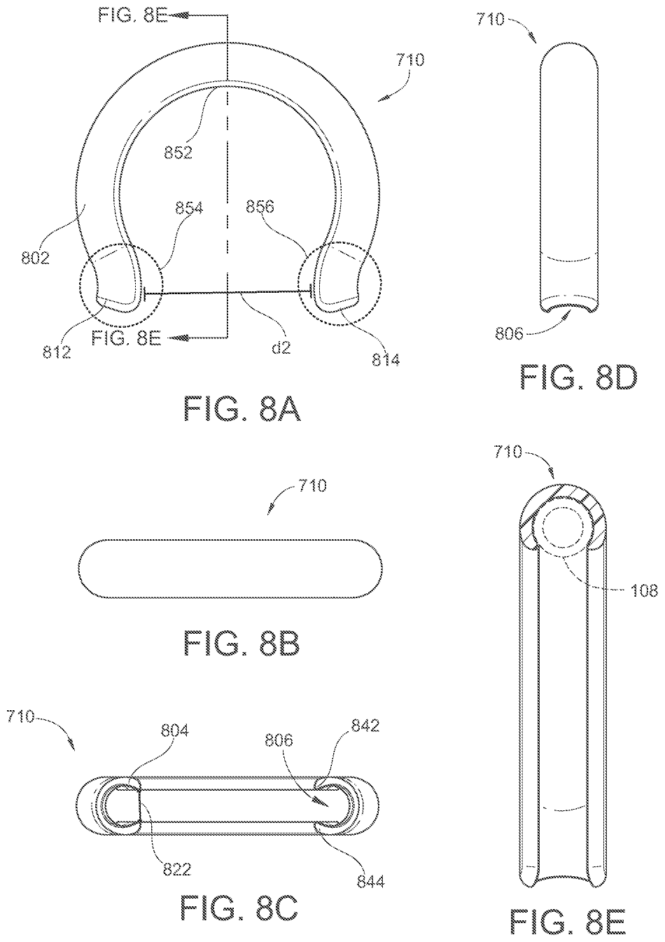

FIGS. 8A-8E illustrate top, back, front, side, and cross-sectional views, respectively, of the tube securement member of FIG. 7A. As compared to the headgear assembly 200 of FIG. 1, the headgear assembly 700 includes a different implementation of the tube securement member 710. The tube securement member 710 includes a curved, elongate body 802. The body 802 includes a receiving region 806 for removably receiving a portion of fluid tube 108. The body 802 and/or receiving region 806 can be configured to secure a portion of fluid tube 108 such that, when the portion of the fluid tube 108 is secured within the receiving region 806, the portion of the fluid tube 108 has a curved configuration. The receiving region 806 can extend (for example, continuously) from a first end 812 of the body 802 to a second end of the body 802. The receiving region 806 can be a channel, for example, extending continuously between such first and second ends 812, 814. The body 802 can include a wall 804 that at least partially defines the receiving region 806. The wall 804 can extend circumferentially around the receiving region 806 (for example, circumferentially around a portion of a cross-section of the receiving region 806). The wall 804 can define a generally radially-inward-facing opening 822 extending along the complete length of the receiving region 806. In this way, the opening 822 at the first end 812 and the opening 822 at the second end 814 can face each other. Although the opening 822 is illustrated as radially-inward-facing, it will be understood that the location of the opening 822 may vary across embodiments. For example, in some cases, the opening 822 is radially outward facing such that the fluid tube 108 can be inserted around an outside or exterior of the tube securement member 710.

The wall 804 can include a first edge 842 and a second edge 844. In some cases, the second edge 844 is inset from the first edge 842. In some cases, the first edge 842 and/or the second edge 844 defines all or a portion of an exterior perimeter of the curved elongate body 802. In some cases, the first edge 842 and/or the second edge 844 defines all or a portion of an interior perimeter of the curved elongate body 802. As shown, in some cases, each of the first edge 842 and the second edge 844 can be an inner rim, in that they face radially inward. Alternatively, in some cases, the first edge 842 and/or the second edge 844 can be an outer rim, in that they face radially outward. The first end 812 and the second end 814 can be separated by a gap, d2. The gap, d2, can vary across embodiments. For example, d2 may range from a few millimeters to a centimeter, from 1 to 3 centimeters, from 2 to 4 centimeters, up to six centimeters, etc. As another example, the size of d2 may be relative to the diameter of the tube securement member 710. For example, d2 may be equal to the diameter of the tube securement member 710. As another example, d2 may be about 75%, about 80%, about 90%, about 95%, about 105%, about 110%, or about 115% of the diameter of the tube securement member 710.

As shown, the tube securement member 710 is an elongate member including a curved central portion 852 and two curved distal end portions 854, 856. As shown, the curve of the curved central portion 852 can oppose a curve of each of the curved distal end portions 854, 856. That is, the curved central portion 852 can curve towards the interior of the tube securement member 710, while the curved distal end portions 854, 856 curved away from the interior of the tube securement member 710. In some cases, the tube securement member 710 may not include the curved distal end portions 854, 856. In addition or alternatively, the tube securement member 710 may include end portions that are relatively straight. In some cases, the receiving region 806 has a curved shape with a partial circular section extending from the first end 812 to the second end 814, and the two curved distal end portions 854, 856 are curved extensions on opposite ends of the partial circular section. Each curved extension can curve in a direction opposite to that of the partial circular section.

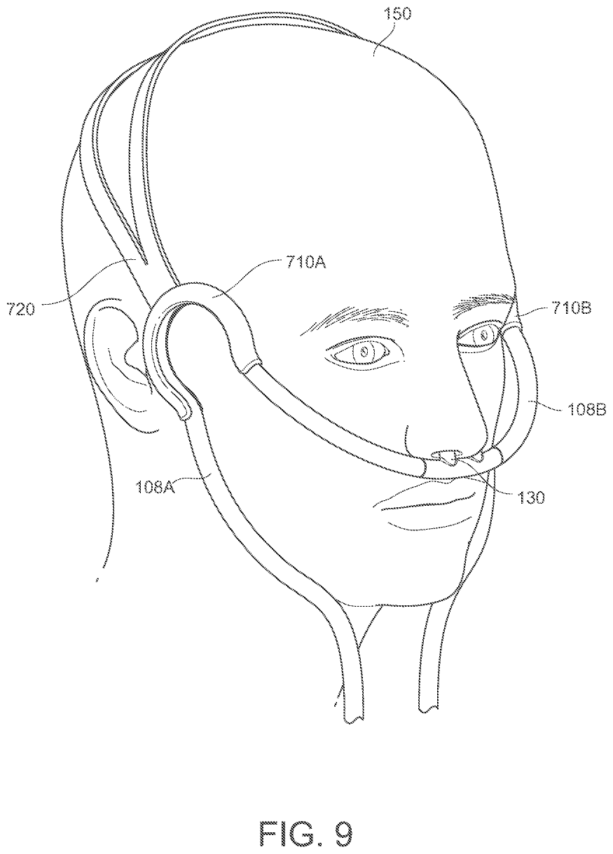

FIG. 9 illustrates an example of the headgear assembly 700 of FIG. 7A coupled to fluid tubes 108 of an example respiratory support system and positioned on a subject 150. As shown, when the headgear assembly 700 and respiratory support device 130 are attached to the subject, the fluid tubes 108 extend from the respiratory support device 130 (in this case, a cannula) to the respective tube securement members 710. The tube securement members 710 curve or bend the fluid tubes 108 down together along the front of the chest. In some implementations, the headgear assembly 700 is configured to secure to a subject's head and secure one or more portions of fluid tubes 108A, 108B (which can be connected to nasal cannula with one or more nasal prongs) such that no portion of the headgear assembly 700 contacts the subject's ears. For example, in some implementations, the headgear assembly 700 is configured to secure to a subject's head and secure one or more portions of fluid tubes 108A, 108B (which can be connected to nasal cannula with one or more nasal prongs) such that no portion of the headband 720 and/or no portion of the tube securement member(s) 710A, 710B (either or both of which can be included in assembly 700) contact the subject's ears. In some implementations, tube securement member(s) 710A, 710B are sized and/or shaped to secure and/or operably position portions of tubes 108A, 108B in a manner that is similar to a manner in which such tubes 108A, 108B would be secured and/or operably positioned if tubes 108A, 108B were wrapped around the subject's ears. For example, in some implementations (such as that illustrated in FIG. 9), tube securement member(s) 710A, 710B are configured to secure portions of tubes 108A, 108B in a curved arrangement that is similar to a curved arrangement that such portions of tubes 108A, 108B would have if wrapped around the subject's ears. In some implementations, the headgear assembly 700 is configured to secure to a subject's head and secure one or more portions of fluid tubes 108A, 108B (which can be connected to nasal cannula with one or more nasal prongs) such that each tube securement member 710A, 710B is positioned: in front of the subject's ear; between the subject's ear and the subject's eye; and/or adjacent the subject's cheek (for example, upper cheek). In some implementations, the headgear assembly 700 is configured to secure to a subject's head and secure one or more portions of fluid tubes 108A, 108B (which can be connected to nasal cannula with one or more nasal prongs) such that tubes 108A, 108B do not contact skin of the subject's face (for example, cheek).

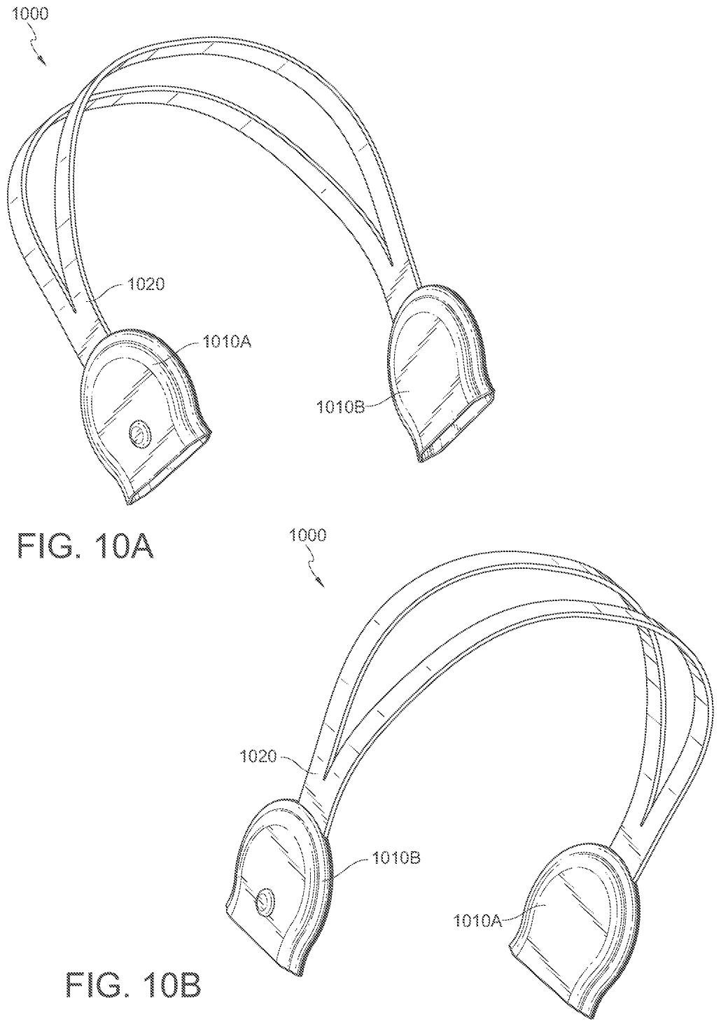

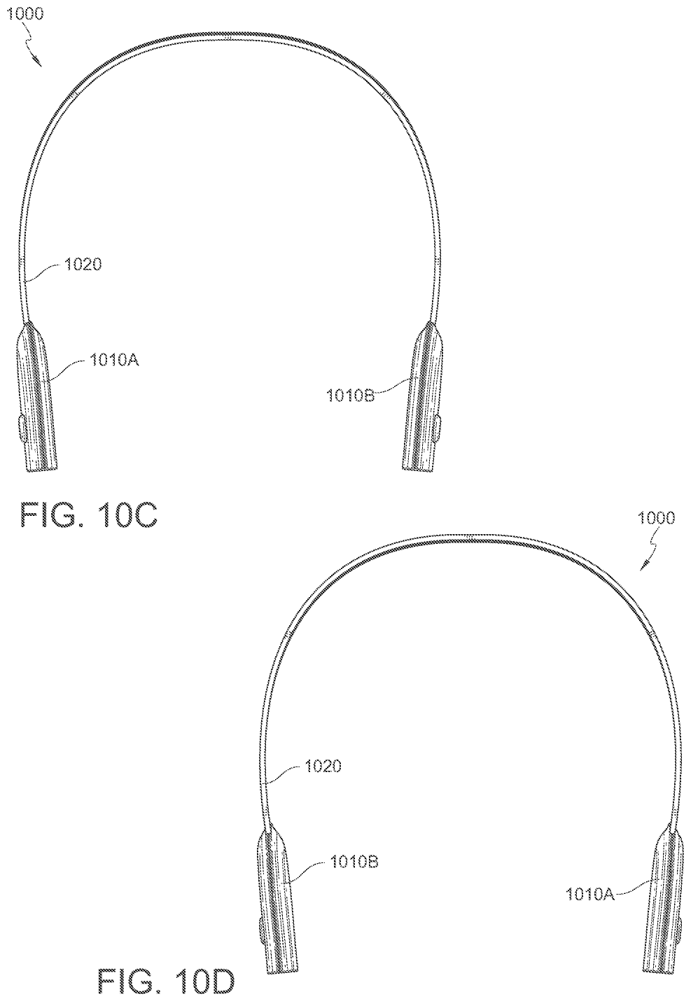

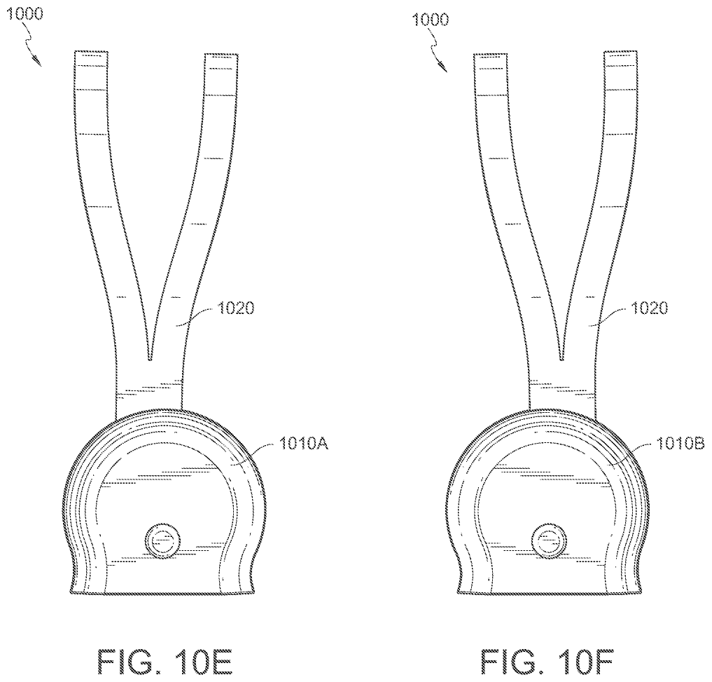

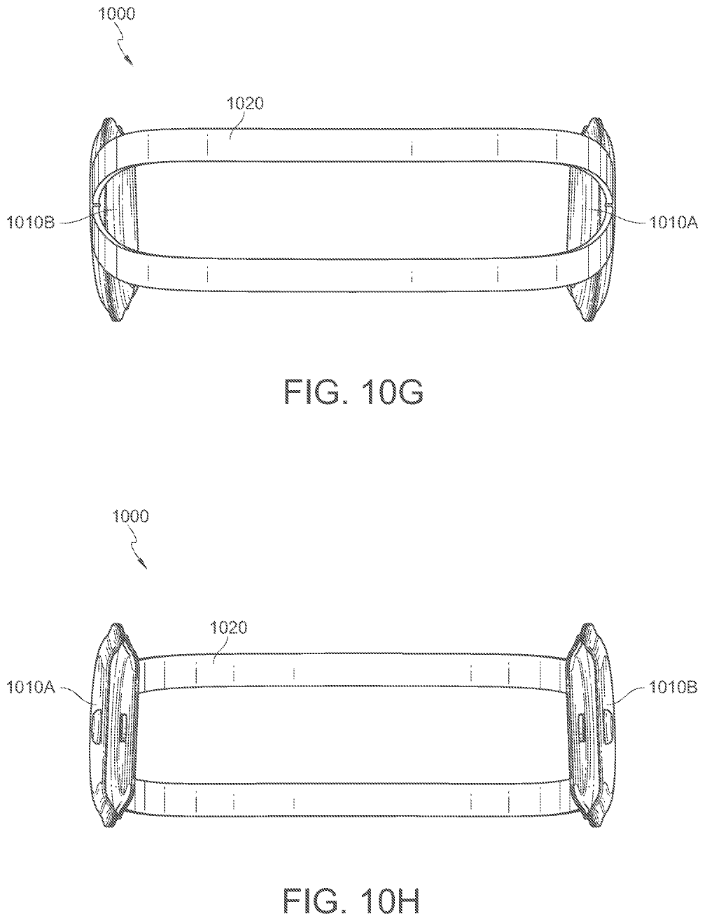

FIGS. 10A-10H depict first perspective, second perspective, front, back, first side, second side, top, and bottom views, respectively, of an example headgear assembly 1000. The headgear assembly 1000 includes a headband 1020 and a plurality of tube securement members 1010A, 1010B coupled to either end of the headband 1020. For ease of description, the plurality of tube securement members 1010A, 1010B are individually or collectively referred to as the tube securement member 1010, the tube securement member(s) 1010, or the tube securement members 1010. The headgear assembly 1000, tube securement members 1010, and/or headband 1020 can be embodiments of the headgear assembly 200, tube securement members 210, and/or headband 220 of FIG. 1.

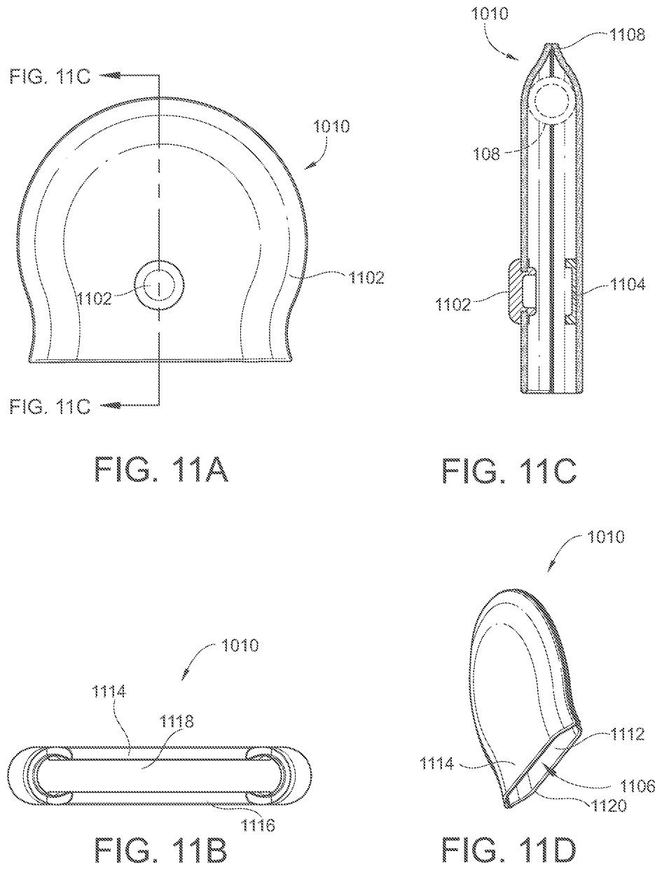

FIGS. 11A-11D illustrate front, bottom, cross-sectional, and back perspective views, respectively, of the tube securement member of FIG. 10A. As compared to the headgear assembly 200 of FIG. 1, the headgear assembly 1000 includes a different implementation of the tube securement member 1010. In particular, the tube securement member 1010 is implemented as a pouch-like member. The tube securement member 1010 can be made any suitable material, such as one or more portions of fabric, a shape memory material, an elastic material, or a rigid material.

The tube securement member 1010 includes a first wall 1112 and a second wall 1116. The first and second walls 1112, 1114 are opposing walls that intersect along an interior surface 1108 to provide a cohesive interior surface 1118 defining a receiving region 1106. Furthermore, the first and second walls 1112, 1114 define an opening 1120 for accessing the receiving region 1106. For example, edges 1122 of the walls 1112, 1114 can define the opening 1120.

To secure a fluid tube 108 into the tube securement member 1010, the fluid tube 108 is flexed to fit through the opening 1120 and enter the receiving region 1106. The fluid tube 108 is then pushed into the receiving region 1106 until the fluid tube 108 aligned with the intersecting interior surface 1108 of the first and second walls 1112, 1114. FIG. 11C illustrates example positioning of the fluid tube 108 within the tube securement member 1010. After the fluid tube 108 is inside the tube securement member 1010, the first engagement member 1102 is activated.

The tube securement member 1010 includes a first engagement member 1102 and a second engagement member 1104 for securing the fluid tube within the tube securement member 1010. In the illustrated example, the first engagement member 1102 is a male portion of a snap press button and the second engagement member 1104 is a female portion of the snap press button. In use, the fluid tube 108 is inserting into the receiving region 1106 when the first and second engagement members 1102, 1104 are not engaged, and then the first and second engagement members 1102, 1104 are engaged to further second the fluid tube 108 to the tube securement member 1010. In some implementations, the first and second engagement members 1102, 1104 can include any coinciding coupling features (e.g., a snap fit pairing, a strap and buckle, a living hinge and coinciding lip, an interference fit pairing, a hook and loop fastener, a protrusion and slot, a clip, a clamp, a clasp, a buckle, a button, a hook and protrusion, a screw and aperture, a wire, etc.). In some cases, more than one coupling feature can be used.

The first engagement member 1102 and the second engagement member 1104 can be positioned on opposite facing walls of the tube securement member 1010. The positioning of the first and second engagement members 1102 along the tube securement member 1010 can vary across embodiments. For instance, in the illustrated example, the first and second engagement members 1102, 1104 are position near a bottom center portion of the tube securement member 1010.

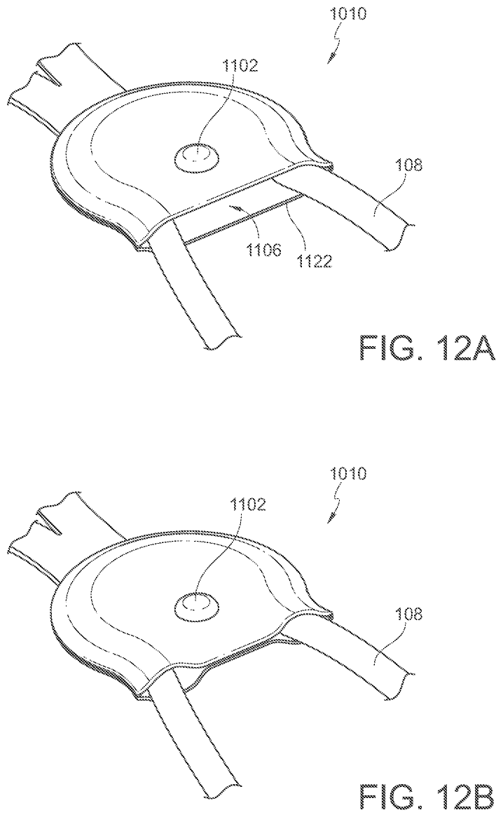

FIGS. 12A and 12B illustrate example open and closed configurations, respectively, of the tube securement member 1010 of FIG. 10A. As described herein, the tube securement member 1010 includes at least a first engagement member 1102 configured to facilitate the closure of the pouch and/or the securement of the fluid tube 108 within the pouch. The example of FIG. 12A illustrates the pouch in an open configuration in which the first engagement member 1102 is not engaged. As such, in this example, the receiving region 1106 is open and available to receive the fluid tube 108 through the opening 1120. In contrast, the example of FIG. 12B illustrates the pouch in a closed configuration in which the first engagement member 1102 is engaged. As such, in this example, the receiving region 1106 is closed, and the likelihood that the fluid tube 108 disconnects from the tube securement member 1010 is reduced.

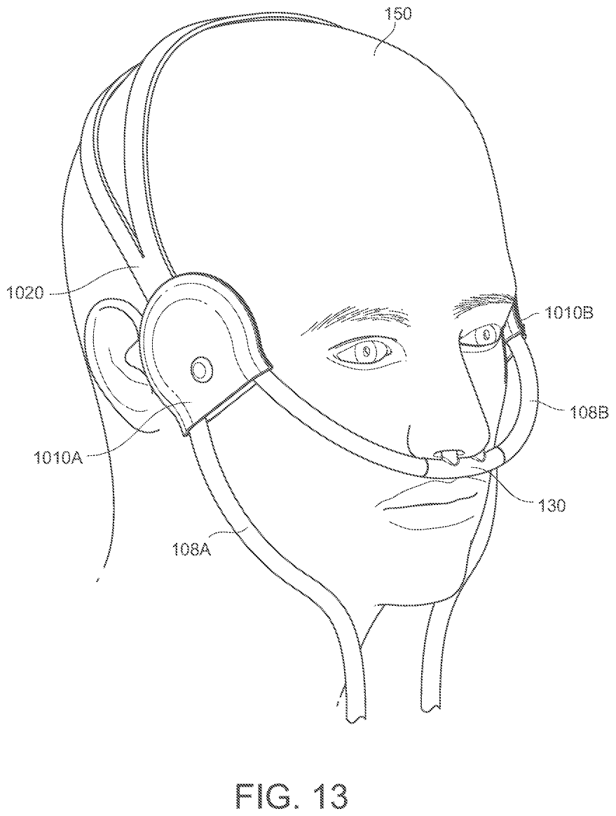

FIG. 13 illustrates a perspective view of the headgear assembly 1000 of FIG. 10A positioned on a subject 150 and coupled to an example respiratory support device 130. As shown, when the headgear assembly 1000 and respiratory support device 150 (in this case, a nasal cannula) are attached to the subject, the fluid tubes 108 extend from the nasal cannula to the respective tube securement members 1010. The tube securement members 1010 curve or bend the fluid tubes 108 down together along the front of the chest. In some implementations, the headgear assembly 1000 is configured to secure to a subject's head and secure one or more portions of fluid tubes 108A, 108B (which can be connected to nasal cannula with one or more nasal prongs) such that no portion of the headgear assembly 1000 contacts the subject's ears. For example, in some implementations, the headgear assembly 1000 is configured to secure to a subject's head and secure one or more portions of fluid tubes 108A, 108B (which can be connected to nasal cannula with one or more nasal prongs) such that no portion of the headband 1020 and/or no portion of the tube securement member(s) 1010A, 1010B (either or both of which can be included in assembly 1000) contact the subject's ears. In some implementations, tube securement member(s) 1010A, 1010B are sized and/or shaped to secure and/or operably position portions of tubes 108A, 108B in a manner that is similar to a manner in which such tubes 108A, 108B would be secured and/or operably positioned if tubes 108A, 108B were wrapped around the subject's ears. For example, in some implementations (such as that illustrated in FIG. 9), tube securement member(s) 1010A, 1010B are configured to secure portions of tubes 108A, 108B in a curved arrangement that is similar to a curved arrangement that such portions of tubes 108A, 108B would have if wrapped around the subject's ears. In some implementations, the headgear assembly 1000 is configured to secure to a subject's head and secure one or more portions of fluid tubes 108A, 108B (which can be connected to nasal cannula with one or more nasal prongs) such that each tube securement member 1010A, 1010B is positioned: in front of the subject's ear; between the subject's ear and the subject's eye; and/or adjacent the subject's cheek (for example, upper cheek). In some implementations, the headgear assembly 1000 is configured to secure to a subject's head and secure one or more portions of fluid tubes 108A, 108B (which can be connected to nasal cannula with one or more nasal prongs) such that tubes 108A, 108B do not contact skin of the subject's face (for example, cheek).

### Example Embodiments

Various example embodiments of head gear assemblies can be found in the following clauses.

Clause 1. A headgear assembly, comprising:
a headband; and
a tube securement member coupled to the headband, the tube securement member comprising a wall having a curved shape, with a first end, a second end, and an interior surface defining a receiving region extending from the first end to the second end, the wall enclosing a majority of a cross-sectional perimeter of the receiving region, the wall defining an opening extending continuously along a length of the receiving region, the opening being defined by a gap between first and second edges of the wall, the first and second edges extending along a length of the wall;
wherein the tube securement member is configured to receive a portion of a fluid tube into the receiving region through the opening and removably secure the portion of the fluid tube in the receiving region.

Clause 2. The headgear assembly of any of the preceding clauses, wherein the first and second edges of the wall are spaced apart by a predetermined distance that is less than a diameter of the receiving region.

Clause 3. The headgear assembly of any of the preceding clauses, wherein the receiving region having a curved shape with a partial circular section extending from the first end of the wall to the second end of the wall and two curved extensions on opposite ends of the partial circular section, each curved extension curving in a direction opposite to that of the partial circular section.

Clause 4. The headgear assembly of clause 3, wherein each curved extension has a radius of curvature that is different from a radius of curvature of the partial circular section.

Clause 5. The headgear assembly of any of the preceding clauses, wherein the tube securement member is configured to removably secure the portion of the fluid tube therein by frictional engagement between the interior surface of the wall and an outer surface of the portion of the fluid tube.

Clause 6. The headgear assembly of any of the preceding clauses, wherein the receiving region has a shape that is generally U-shaped or generally C-shaped.

Clause 7. The headgear assembly of any of the preceding clauses, wherein the first and second edges of the wall each include a lip that extends toward the interior surface of the wall, the lips configured to apply a compressive force to the portion of the fluid tube to aid retainment of the portion of the fluid tube in the receiving region.

Clause 8. The headgear assembly of any of the preceding clauses, wherein tube securement member has a face contacting side and a non-face contacting side, wherein the opening is located on the non-face contacting side of the tube securement member.

Clause 9. The headgear assembly of any of the preceding clauses, wherein the opening extends radially inward.

Clause 10. The headgear assembly of any of the preceding clauses, wherein the wall extends circumferentially around the receiving region.

Clause 11. The headgear assembly of any of the preceding clauses, wherein the tube securement member is a first tube securement member, wherein the headgear assembly further comprises a second tube securement member for removably securing a second portion of the fluid tube, wherein, when coupled to the fluid tube, the first tube securement member and the second tube securement member cooperate to properly position prongs of a nasal cannula in nostrils of a subject.

Clause 12. The headgear assembly of any of the preceding clauses, wherein the tube securement member is a first tube securement member, wherein the headgear assembly further comprises a second tube securement member for removably securing a second portion of the fluid tube, wherein the first and second tube securement members are configured to be placed on opposite sides of a head of a subject, with the first and second tube securement members cooperating to support the fluid tube during use.

Clause 13. The headgear assembly of any of the preceding clauses, wherein the tube securement member is configured to receive and secure the portion of the fluid tube in a curved arrangement to inhibit kinking of the fluid tube.

Clause 14. The headgear assembly of any of the preceding clauses, wherein, when properly positioned on a head of a subject and properly coupled to the fluid tube, neither the headgear assembly nor the fluid tube contacts any portion of either ear of the subject.

Clause 15. The headgear assembly of any of the preceding clauses, wherein the tube securement member further comprises an engagement member, wherein the engagement member is configured to engage with a complementary engagement member of the headband to couple the headband to the tube securement member.

Clause 16. A headgear assembly, comprising:
a headband; and
a tube securement member coupled to the headband, the tube securement member comprising opposing walls intersecting along an interior surface to provide a cohesive interior surface defining a receiving region, wherein edges of the walls define an opening, and
wherein the tube securement member is configured to receive a portion of a fluid tube into the receiving region through the opening and removably secure the portion of the fluid tube.

Clause 17. The headgear assembly of clause 16, wherein the tube securement member is a pouch-like member, and wherein the walls are flexible walls.

Clause 18. The headgear assembly of any of clauses 16 or 17, wherein the opposing walls each comprise an engagement member that are matable with each other to selectively close a portion of the opening.

Clause 19. The headgear assembly of any of clauses 16-18, wherein the tube securement member is a first tube securement member, wherein the headgear assembly further comprises a second tube securement member for removably securing a second portion of the fluid tube, wherein, when coupled to the fluid tube, the first tube securement member and the second tube securement member cooperate to properly position prongs of a nasal cannula in nostrils of a subject.

Clause 20. A respiratory support device system comprising:
a fluid management apparatus configured to deliver therapeutic fluids to and/or collect fluids from a subject;
a nasal cannula comprising prongs for placement in nostrils of the subject;
a fluid tube fluidically connected to the fluid management apparatus and the nasal cannula; and
a headgear assembly configured to secure the fluid tube and properly position the prongs of the nasal cannula in the nostrils of the subject, the headgear assembly comprising:
a headband; and
a headgear assembly comprising:
a headband, and
a pair of tube securement member coupled to opposite ends of the headband, each tube securement member comprising a wall having a curved shape, with a first end, a second end, and an interior surface defining a receiving region extending from the first end to the second end, the wall enclosing a majority of a cross-sectional perimeter of the receiving region, the wall defining an opening extending continuously along a length of the receiving region, the opening being defined by a gap between first and second edges of the wall, the first and second edges extending along a length of the wall,
wherein the tube securement members are configured to receive portions of the fluid tube into the respective receiving regions through the respective openings and removably secure the respective portions of the fluid tube in the respective receiving regions.

Clause 21. A headgear assembly comprising:
a tube securement member comprising a curved elongate body, wherein the curved elongate body comprises a wall defining a receiving region extending from a first end portion of the curved elongate body to a second end portion of the curved elongate body, wherein the wall extends circumferentially around the receiving region and defines a gap extending along a length of the receiving region, wherein the receiving region removably receives a portion of a fluid tube through the gap; and
an attachment member coupled to the tube securement member and configured to secure the tube securement member to a head of a subject.

Clause 22. The headgear assembly of clause 21, wherein the tube securement member is configured to receive and secure the portion of the fluid tube in a curved arrangement to inhibit kinking of the fluid tube.

Clause 23. The headgear assembly of any of clauses 21 or 22, wherein the receiving region is configured to secure the portion of fluid tube such that, when the portion of the fluid tube is secured within the receiving region, the portion of the fluid tube has a curved configuration.

Clause 24. The headgear assembly of any of clauses 21 to 23, wherein the receiving region is a curved channel formed by the wall of the curved elongate body.

Clause 25. The headgear assembly of any of clauses 21 to 24, wherein the receiving region extends continuously between the first and second end portions.

Clause 26. The headgear assembly of any of clauses 21 to 25, wherein the receiving region is generally cylindrical.

Clause 27. The headgear assembly of any of clauses 21 to 26, wherein the receiving region is defined by a generally cylindrical exterior structure that is curved along its length.

Clause 28. The headgear assembly of any of clauses 21 to 27, wherein the receiving region extends along a complete length of the body.

Clause 29. The headgear assembly of any of clauses 21 to 28, wherein the receiving region does not fully enclose any portions of the fluid tube.

Clause 30. The headgear assembly of any of clauses 21 to 29, wherein the wall includes an outer rim and an inner rim, wherein the outer rim and the inner rim are separated by the gap.

Clause 31. The headgear assembly of any of clauses 21 to 30, wherein the wall includes an outer rim and an inner rim, wherein the inner rim is inset from the outer rim.

Clause 32. The headgear assembly of any of clauses 21 to 31, wherein the wall includes an outer rim and an inner rim, wherein the outer rim defines a portion of an exterior perimeter of the curved elongate body.

Clause 33. The headgear assembly of any of clauses 21 to 32, wherein the wall includes an outer rim and an inner rim, wherein the inner rim is higher than the outer rim.

Clause 34. The headgear assembly of any of clauses 21 to 33, wherein the wall includes an outer rim and an inner rim, wherein the outer rim is higher than the inner rim.

Clause 35. The headgear assembly of any of clauses 21 to 34, wherein the tube securement member is a first tube securement member, wherein the headgear assembly further comprises a second tube securement member coupled to the attachment member.

Clause 36. The headgear assembly of clause 35, wherein when the headgear assembly is positioned on the head, the first and second tube securement members are positioned on opposite sides of the head and adjacent to opposing ears of the subject.

Clause 37. The headgear assembly of any of clauses 21 to 36, wherein the tube securement member further comprises an engagement member, wherein the engagement member is configured to engage with a complementary engagement member of the attachment member to couple the attachment member to the tube securement member.

Clause 38. The headgear assembly of clause 37, wherein the engagement member is integrally formed with the curved elongate body.

Clause 39. The headgear assembly of any of clauses 21 to 38, wherein the headgear assembly is securable to the head of the subject without using either ear of the subject as support.

Clause 40. The headgear assembly of any of clauses 21 to 39, wherein the headgear assembly is securable to the head of the subject without contacting either ear of the subject.

Clause 41. The headgear assembly of any of clauses 21 to 40, wherein when the headgear assembly is positioned on the head of the subject, the tube securement member is positioned in front of an ear.

Clause 42. The headgear assembly of any of clauses 21 to 41, wherein when the headgear assembly is positioned on the head of the subject, the tube securement member is positioned behind an ear.

Clause 43. The headgear assembly of any of clauses 21 to 42, wherein when the headgear assembly is positioned on the head of the subject, the tube securement member is positioned above an ear.

Clause 44. The headgear assembly of any of clauses 21 to 43, wherein the tube securement member is at least one of generally U-shaped or generally C-shaped.

Clause 45. The headgear assembly of any of clauses 21 to 44, wherein the gap is an outward facing gap such that when the headgear assembly is positioned on the head of the subject, the gap faces away from the subject.

Clause 46. The headgear assembly of any of clauses 21 to 45, wherein the gap is an inward facing gap such that when the headgear assembly is positioned on the head of the subject, the gap faces towards the subject.

Clause 47. The headgear assembly of any of clauses 21 to 46, wherein the gap faces radially inward.

Clause 48. The headgear assembly of any of clauses 21 to 47, wherein the one or more disconnection-resisting features includes a portion of the gap that is sized smaller than a diameter of the fluid tube.

Clause 49. The headgear assembly of any of clauses 21 to 48, wherein the tube securement member further comprises one or more disconnection-resisting features configured to resist disconnection between the tube securement member and the portion of the fluid tube.

Clause 50. The headgear assembly of any of clauses 21 to 49, wherein the headgear assembly facilitates proper positioning of a nasal cannula without using either ear as support.

Clause 51. The headgear assembly of any of clauses 21 to 50, wherein the tube securement member is made of resilient, shape-memory material.

Clause 52. The headgear assembly of any of clauses 21 to 51, wherein the tube securement member is a unitary structure.

Clause 53. The headgear assembly of any of clauses 21 to 52, wherein the tube securement member and the attachment member are integrally formed.

Clause 54. The headgear assembly of any of clauses 21 to 53, wherein each of the first and second end portions comprises an open face.

Clause 55. The headgear assembly of any of clauses 21 to 54, wherein the tube securement member comprises a curved central portion and two curved end portions, wherein a curve of the curved central portion opposes a curve of each of the curved end portions.

Clause 56. The headgear assembly of any of clauses 21 to 55, wherein the tube securement member secures the fluid such that the fluid tube is not kinked.

Clause 57. The headgear assembly of any of clauses 21 to 56, wherein the attachment member comprises a contoured surface having a contour that is substantially matched to a shape of the head of the subject.

Clause 58. The headgear assembly of any of clauses 21 to 57, wherein the attachment member is a resilient open-loop headband, the open-loop headband having an arcuately contoured one-piece configuration terminating at a pair of legs for resiliently capturing therebetween the head of the subject.

Clause 59. The headgear assembly of any of clauses 21 to 58, wherein the headgear assembly is reusable.

Clause 60. A headgear assembly for securing a fluid tube to a subject, the headgear assembly comprising:
a tube securement member, wherein the tube securement member comprises first and second opposing walls intersecting along an interior surface to provide a cohesive interior surface defining a receiving region, wherein the first and second opposing walls define a gap for accessing the receiving region, wherein the receiving region removably receives a portion of a fluid tube through the gap; and
an attachment member coupled to the tube securement member and configured to secure the tube securement member to a head of a subject.

Clause 61. The headgear assembly of clause 60, wherein the tube securement member is implemented as a pouch.

Clause 62. The headgear assembly of any of clauses 60 or 61, further comprising a plurality of engagement members configured to mate to close at least a portion of the receiving region.

Clause 63. The headgear assembly of clause 62, further comprising any combination of components or features of any of the previous clauses.

Clause 64. A tube securement member of a headgear assembly for securing a fluid tube to a subject, the tube securement member comprising:
a curved elongate body comprising a wall defining a receiving region extending from a first end portion of the curved elongate body to a second end portion of the curved elongate body, wherein the wall extends circumferentially around the receiving region and defines a gap extending along a length of the receiving region, wherein the receiving region removably receives a portion of a fluid tube through the gap.

Clause 65. The tube securement member of clause 64, further comprising any combination of components or features of any of the previous clauses.

Clause 66. An assembly for securing a fluid tube to a subject's head, the assembly comprising:
an attachment member configured to removably secure to at least a portion of the subject's head; and
tube securement member coupled to the attachment member and configured to be secured to the subject's head by the attachment member, the tube securement member configured to receive and secure a portion of a fluid tube in a curved arrangement to inhibit kinking of the tube.

Clause 67. The assembly of clause 66, further comprising any combination of components or features of any of the previous clauses.

Clause 68. A nasal cannula system comprising:
a fluid management apparatus configured to deliver therapeutic fluids to and/or collect fluids from a subject;
a nasal canula comprising prongs for placement in nostrils of the subject;
a fluid tube fluid connected to the fluid management apparatus and the nasal canula; and
a headgear assembly for securing the fluid tube and the nasal cannula to the subject, wherein the headgear assembly comprises any one or any combination of the features of the headgear assembly according to any of the previous clauses.

Clause 69. A method of attaching a headgear assembly for securing a fluid tube to a subject, the method comprising:
providing the headgear assembly, the headgear assembly comprising an attachment member configured to secure the tube securement member to a head of a subject, the headgear assembly further comprising a tube securement member coupled to the attachment member, wherein the a tube securement member comprises a curved elongate body, wherein the curved elongate body comprises a wall defining a receiving region extending from a first end portion of the curved elongate body to a second end portion of the curved elongate body, wherein the wall extends circumferentially around the receiving region and defines a gap extending along a length of the receiving region, wherein the receiving region removably receives a portion of a fluid tube through the gap;
providing a nasal cannula system, the nasal cannula system comprising a fluid tube;
securing the fluid tube to the tube securement member, wherein said securing comprises inserting a middle portion of the fluid tube into receiving region through the gap.

Clause 70. A headgear assembly for securing a nasal cannula to a subject, the headgear assembly comprising:
a headband configured to be wrapped around a portion of a head of the subject; and
a tube securement member coupled to the headband, the tube securement member comprising a channel having a first end and a second end, said channel being curved between the first and second ends and defining a receiving region configured to secure a portion of a tube connected to the nasal cannula, wherein said channel is configured to face away from skin of the subject when the headgear assembly is in use.

Clause 71. The headgear assembly of clause 70, wherein the headgear assembly is configured to secure the first tube connected to the nasal cannula to the subject's head such that no portion of the first tube contacts the subject's ears.

Clause 72. The headgear assembly of clause 71, wherein the headgear assembly is configured to secure the first tube connected to the nasal cannula to the subject's head such that no portion of the tube securement member contacts the subject's ears.

Clause 73. The headgear assembly of any of clauses 70 to 72, wherein the channel of the tube securement member is formed by a wall having a curved cross-section, said wall having a first edge and a second edge that are separated from one another by a gap, said gap at least partially defining an opening into said receiving region.

Clause 74. The headgear assembly of clause 73, wherein said first edge extends along a first plane and said second edge extends along a second plane that is spaced from said first plane.

Clause 75. The headgear assembly of clause 73 or 74, wherein said second edge is inset from said first edge.

Clause 76. The headgear assembly of any of clauses 73 to 75, wherein said gap is less than a diameter of the curved cross-section of the wall.

Clause 77. The headgear assembly of any of clauses 70 to 76, wherein the tube securement member is configured to removably secure the portion of the tube within said receiving region by frictional engagement.

Clause 78. The headgear assembly of any of clauses 70 to 77, wherein the channel is generally C-shaped.

Clause 79. The headgear assembly of any of clauses 70 to 78, wherein the headgear assembly secures the portion of the tube without contacting the nasal cannula.

Clause 80. The headgear assembly of any of clauses 70 to 79, wherein the tube securement member further comprises an engagement member comprising a slot configured to allow a portion of the headband to pass therethrough and secure to itself.

Clause 80. The headgear assembly of the preceding clauses, wherein:
the tube securement member is a first tube securement member of the headgear assembly, said receiving region of said first tube securement member configured to receive a portion of a first tube connected to the nasal cannula; and
the headgear assembly further comprises a second tube securement member coupled to the headband, the second tube securement member comprising a channel having a first end and a second end, said channel of said second tube securement member being curved between the first and second ends of the second tube securement member and defining a receiving region configured to receive a portion of a second tube connected to the nasal cannula.

Clause 81. A system comprising the headgear assembly of any of the preceding clauses and further comprising the nasal cannula and said tube connected to the nasal cannula.

Clause 82. A headgear assembly for securing a nasal cannula to a subject's nostrils, the headgear assembly comprising:
a headband configured to be wrapped around a portion of a head of the subject;
a first tube securement member connected to a first portion of the headband, the first tube securement member comprising a curved channel configured to removably receive a first tube connected to the nasal cannula; and
a second tube securement member connected to a second portion of the headband and separate from said first tube securement member, the second tube securement member comprising a curved channel configured to removably receive a second tube connected to the nasal cannula;
wherein the headgear assembly is configured to secure the first and second tubes connected to the nasal cannula to the subject's head such that no portion of the first and second tubes contact ears of the subject.

Clause 83. The headgear assembly of Clause 82, wherein each of the curved channels of the first and second tube securement members is formed by a wall having a curved cross-section, said wall having a first edge and a second edge that are separated from one another by a gap, said gap at least partially defining an opening into a receiving region defined by said channel.

### Terminology

Any or all of the features and functions described above can be combined with each other, except to the extent it may be otherwise stated above or to the extent that any such embodiments may be incompatible by virtue of their function or structure, as will be apparent to persons of ordinary skill in the art. Unless contrary to physical possibility, it is envisioned that the methods/steps described herein may be performed in any sequence and/or in any combination, and the components of respective embodiments may be combined in any manner.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims, and other equivalent features and acts are intended to be within the scope of the claims.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without subject input or prompting, whether these features, elements and/or steps are included or are to be performed in any particular embodiment.

Unless the context clearly requires otherwise, throughout the description and the claims, the words “comprise,” “comprising,” and the like are to be construed in an inclusive sense, as opposed to an exclusive or exhaustive sense, e.g., in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words using the singular or plural number may also include the plural or singular number, respectively. The word “or” in reference to a list of two or more items, covers all of the following interpretations of the word: any one of the items in the list, all of the items in the list, and any combination of the items in the list. Likewise, the term “and/or” in reference to a list of two or more items, covers all of the following interpretations of the word: any one of the items in the list, all of the items in the list, and any combination of the items in the list.

Conjunctive language such as the phrase “at least one of X, Y and Z,” unless specifically stated otherwise, is otherwise understood with the context as used in general to convey that an item, term, etc. may be either X, Y or Z, or any combination thereof. Thus, such conjunctive language is not generally intended to imply that certain embodiments require at least one of X, at least one of Y and at least one of Z to each be present. Further, use of the phrase “at least one of X, Y or Z” as used in general is to convey that an item, term, etc. may be either X, Y or Z, or any combination thereof.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

Any terms generally associated with circles, such as “radius” or “radial” or “diameter” or “circumference” or “circumferential” or any derivatives or similar types of terms are intended to be used to designate any corresponding structure in any type of geometry, not just circular structures. For example, “radial” as applied to another geometric structure should be understood to refer to a direction or distance between a location corresponding to a general geometric center of such structure to a perimeter of such structure; “diameter” as applied to another geometric structure should be understood to refer to a cross sectional width of such structure; and “circumference” as applied to another geometric structure should be understood to refer to a perimeter region. Nothing in this specification or drawings should be interpreted to limit these terms to only circles or circular structures.

Any patents and applications and other references noted above, including any that may be listed in accompanying filing papers, are incorporated herein by reference. Aspects of the invention can be modified, if necessary, to employ the systems, functions, and concepts of the various references described above to provide yet further implementations of the invention. These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

To reduce the number of claims, certain aspects of the invention are presented below in certain claim forms, but the applicant contemplates other aspects of the invention in any number of claim forms. Any claims intended to be treated under 35 U.S.C. § 112(f) will begin with the words “means for,” but use of the term “for” in any other context is not intended to invoke treatment under 35 U.S.C. § 112(f). Accordingly, the applicant reserves the right to pursue additional claims after filing this application, in either this application or in a continuing application.

What is claimed is:

1. A headgear assembly for securing a nasal cannula to a subject, the headgear assembly comprising:
a headband configured to be wrapped around a portion of a head of the subject; and
a tube securement member coupled to the headband, the tube securement member comprising a channel having a first end and a second end, the channel being curved between the first and second ends and defining a receiving region configured to secure a portion of a tube connected to the nasal cannula,
wherein the channel of the tube securement member is formed by a wall having a curved cross-section, the wall having a first edge and a second edge that are separated from one another by a gap, the gap at least partially defining an opening into the receiving region,
wherein the channel is configured to face away from skin of the subject when the headgear assembly is in use, and
wherein the headgear assembly is configured to secure the portion of the tube connected to the nasal cannula to the subject's head such that no portion of the tube and no portion of the tube securement member contacts the subject's ears.

2. A headgear assembly for securing a nasal cannula to a subject, the headgear assembly comprising:
a headband configured to be wrapped around a portion of a head of the subject; and
a tube securement member coupled to the headband, the tube securement member comprising a channel having a first end and a second end, said channel being curved between the first and second ends and defining a receiving region configured to secure a portion of a tube connected to the nasal cannula, wherein said channel is configured to face away from skin of the subject when the headgear assembly is in use.

3. The headgear assembly of claim 2, wherein the headgear assembly is configured to secure the portion of the tube connected to the nasal cannula to the subject's head such that no portion of the tube contacts the subject's ears.

4. The headgear assembly of claim 3, wherein the headgear assembly is configured to secure the portion of the tube connected to the nasal cannula to the subject's head such that no portion of the tube securement member contacts the subject's ears.

5. The headgear assembly of claim 2, wherein the channel of the tube securement member is formed by a wall having a curved cross-section, said wall having a first edge and a second edge that are separated from one another by a gap, said gap at least partially defining an opening into said receiving region.

6. The headgear assembly of claim 5, wherein said first edge extends along a first plane and said second edge extends along a second plane that is spaced from said first plane.

7. The headgear assembly of claim 5, wherein said second edge is inset from said first edge.

8. The headgear assembly of claim 5, wherein said gap is less than a diameter of the curved cross-section of the wall.

9. The headgear assembly of claim 2, wherein the tube securement member is configured to removably secure the portion of the tube within said receiving region by frictional engagement.

10. The headgear assembly of claim 2, wherein the channel is generally C-shaped.

11. The headgear assembly claim 1, wherein the headgear assembly secures the portion of the tube without contacting the nasal cannula.

12. The headgear assembly of claim 2, wherein the tube securement member further comprises an engagement member comprising a slot configured to allow a portion of the headband to pass therethrough and secure to itself.

13. The headgear assembly of claim 2, wherein:
the tube securement member is a first tube securement member of the headgear assembly, said receiving region of said first tube securement member configured to receive a portion of a first tube connected to the nasal cannula; and
the headgear assembly further comprises a second tube securement member coupled to the headband, the second tube securement member comprising a channel having a first end and a second end, said channel of said second tube securement member being curved between the first and second ends of the second tube securement member and defining a receiving region configured to receive a portion of a second tube connected to the nasal cannula.

14. The headgear assembly of claim 2, further comprising the nasal cannula and said tube connected to the nasal cannula.

15. A headgear assembly for securing a nasal cannula to a subject's nostrils, the headgear assembly comprising:
a headband configured to be wrapped around a portion of a head of the subject;
a first tube securement member connected to a first portion of the headband, the first tube securement member comprising a curved channel configured to removably receive a first tube connected to the nasal cannula; and
a second tube securement member connected to a second portion of the headband and separate from said first tube securement member, the second tube securement member comprising a curved channel configured to removably receive a second tube connected to the nasal cannula;
wherein the headgear assembly is configured to secure the first and second tubes connected to the nasal cannula to the subject's head such that no portion of the first and second tubes contact ears of the subject.

16. The headgear assembly of claim 15, wherein each of the curved channels of the first and second tube securement members is formed by a wall having a curved cross-section, said wall having a first edge and a second edge that are separated from one another by a gap, said gap at least partially defining an opening into a receiving region defined by said channel.
